(19) United States
(12) Patent Application Publication
(10) Pub. No.: US 2004/0096433 A1
(43) Pub. Date: May 20, 2004

(54) Regulation of angiontensin II receptors in mammals subject to fetal programming

(76) Inventors: Bennett Hermann Heinrich Breier, Auckland (NZ); Mark Hedley Vickers, Auckland (NZ)

Correspondence Address: FLIESLER MEYER, LLP, FOUR EMBARCADERO CENTER, SUITE 400, SAN FRANCISCO, CA, 94111, US

(21) Appl. No.: 10/643,450

(22) Filed: Aug. 19, 2003

(30) Foreign Application Priority Data
Dec. 11, 2000 (NZ) 508,779
Aug. 19, 2002 (NZ) 520,866

(51) Int. Cl. A61K 48/00; A61K 38/30

(52) U.S. Cl. 424/93.21; 514/12

(57) ABSTRACT

Embodiments of this invention include methods for alleviating hypertension associated with fetal malnutrition in utero, resulting in a post-natal condition known as fetal programming. Factors that lead to fetal programming can be used to predict development of conditions associated with fetal programming. Fetal programming is associated with numerous metabolic consequences, and is also associated with postnatal hypertension. Insulin-like growth factor-1 (IGF-1), analogs of IGF-1 or a compound that can increase the effective concentration of IGF-1 can decrease expression of antiotensin II type 1 receptors in the kidney, and can result in decreased hypertension associated with fetal programming. Use of IGF-1 as either a primary or an adjunct therapy can therefore be used to decrease adverse consequences of hypertension in animals subject to fetal programming.

### RELATED APPLICATIONS

This application is a Continuation-in-Part of U.S. application Ser. No. 10/450,232, filed Jun. 10, 2003 and claims priority to New Zealand Provisional Patent Specification Serial No: 520,886, filed Aug. 19, 2002. Each of the above applications is herein incorporated fully by reference.

### FIELD OF THE INVENTION

This invention relates to regulation of angiotensin type II (A II) receptors in animals subjected to fetal programming. Particularly, this invention relates to regulation of A II receptors by insulin-like growth factor-1 (IGF-I). More particularly, this invention relates to therapeutic and/or prophylactic use of IGF-I to modulate the density, distribution and the potential for signal transduction of A II receptors or A II-like G protein-coupled receptors.

### BACKGROUND

There is increasing evidence that metabolic disorders that manifest in adult life have their roots before birth. This concept of fetal programming is based on epidemiological and experimental observations of close associations between an adverse intrauterine environment and the later onset of adult metabolic and cardiovascular disorders. “Fetal programming” is herein defined as an adaptive process to an adverse intrauterine environment that alters the fetal metabolic and hormonal milieu, resulting in resetting of developmental processes to ensure fetal survival. The persistence of these adaptive responses, designed for survival in a fetal environment, into postnatal life, leads to metabolic and cardiovascular disorders. One disorder is hypertension.

We have developed an animal model of fetal programming where we apply maternal undernutrition throughout gestation, generating a nutrient-deprived intrauterine environment that results in fetal growth retardation, postnatal growth failure and to changes in allometric growth patterns and endocrine parameters of the somatotrophic axis (1, 2). We have recently shown in our animal model that programmed offspring show profound hyperphagia and obesity, hypertension, hyperinsulinism and hyperleptinemia during adult life and that postnatal hypercaloric nutrition amplifies the metabolic and cardiovascular abnormalities induced by fetal programming (3). Thus, this animal model closely resembles the clinical and metabolic abnormalities seen in humans born of low birth weight and furthermore, displays the phenotype described for the clinical association between hypertension, hyperinsulinemia, dyslipidemia, obesity, and cardiovascular disease, known as Syndrome X. Epidemiological studies have shown that those born of low birth weight have increased rates of obesity in adult life (4). This was most clearly shown in a recent report from the Dutch Famine Study where poor nutrition in the first trimester of pregnancy resulted in increased rates of obesity during adult life (5). Animal studies have also shown that maternal malnutrition during pregnancy results in the development of adult-onset obesity in offspring (4,6,7).

Profound hyperphagia is a consequence of programming and a key contributing factor in adult pathogenesis. Food intake in programmed offspring is significantly elevated at an early postnatal age and increases further with advancing age (3). Our studies also suggest that an adverse intrauterine environment can trigger permanent dysregulation of endocrine systems that regulate food intake and energy homeostasis leading to increased adiposity, hypertension, hyperinsulinism and hyperleptinemia.

Angiotensin II (AII) and its receptors have been implicated in the development of certain diseases associated with fetal programming. These AII-mediated diseases include hypertension, cardiac insufficiency, ischemic peripheral circulation disturbances, myocardial ischemia, vein insufficiency, progressive cardiac insufficiency after myocardial infraction, diabetic nephritides, nephritis, arteriosclerosis, hyperaldosteronism, dermatosclerosis, glomerulosclerosis, renal insufficiency, diseases of central nervous system, sensory disturbances including Alzheimer's disease, deficiency of memory, depression, amnesia, senile dementia, anxiety neurosis, catatonia or indisposition, glaucoma, and intraocular hypertension. It is well accepted that AT₁ receptor stimulation contributes to development of hypertension (62) and that AT₁ blockade in patients with hypertension not only reduces blood pressure, but also improves arterial compliance (63, 55).

A group of antihypertensive drugs, ANG II antagonists, has been developed which comprises substances that bind to, but do not result in the activation of AT₁ receptors (AT₁R). Presently ANG II antagonists like candesartan, irbesartan, losartan, telmisartan, valsartan and the like are selective for AT₁R. Inhibition of the rennin-angiotensin system (RAS) by angiotensin-converting enzyme (ACE) inhibition or blockade of AT₁Rs can reduce blood pressure (BP), and exerts a BP-independent renoprotective effect. (43). Selective ANG II receptor antagonism has been show to reduce insulin resistance and improve glucose tolerance. (51).

Insulin-like growth factor-I (IGF-I) is an important regulator of growth, and IGF-I deficiency is associated with prenatal and postnatal growth failure (8, 9). Under conditions of adequate nutrition, IGF-I has been shown to promote postnatal catch-up growth in rats with intrauterine growth retardation (IUGR) caused by gestational protein deficiency (10). IGF-I therapy is associated with increased insulin sensitivity in normal subjects as well as in patients with growth hormone deficiency, type 2-diabetes mellitus and type A insulin-resistance (11). IGF-I can reduce hyperglycemia in patients with severe insulin resistance by direct effects mediated via the IGF-I receptor (12). Hyperglycemia, hyperinsulinemia, and insulin resistance cause vascular disease in type 2 diabetes. IGF-I infusions lower insulin and lipid levels in healthy humans, and reduces plasma leptin concentrations in rats (13), suggesting that IGF-I may reduce the degree of insulin resistance in type 2 diabetes, obesity and hyperlipidemia (14). However, little is known about the effect of IGF-I on appetite. Infusion of IGF-I has been shown to reduce appetite in tumour-bearing rats (15) but a recent study showed no effect on food intake following IGF-I treatment in normal rats, despite the plasma leptin-lowering effects of IGF-I in that study (13).

Clinical studies relating to IGF-I in hypertension are limited but IGF-I has previously been shown to have vasodilatory effects and to improve cardiac function in healthy volunteers (16). Animal studies suggest a role for IGF-I as a mediator of cardiac hypertrophic responses (17).

The effects of IGF-I on cardiovascular and metabolic homeostasis may be modulated by the insulin-like growth factor binding proteins (IGFBPs). IGFBP-1 and 2 levels closely reflect changes related to nutrition, insulin secretion and disease states such as obesity and type 2 diabetes. IGFBP-3 correlates with IGF-I and is a chronic indicator of GH-dependent growth status (18) while IGFBP-4 appears to inhibit IGF actions under most, if not all, experimental conditions (19). Previous work (1, 20 21) has shown differential expression of IGFBPs following fetal growth retardation. However, to date, there are no data on the effect of IGF-I treatment on IGFBPs in postnatal life following fetal programming alone or in combination with hypercaloric nutrition.

This invention provides methods of modulating the density, distribution and the potential for signal transduction of ANG II receptors or ANG II-like G-protein-coupled seven transmembrane receptors expressed in mammalian tissue. Methods provided in the invention may be used as an independent treatment or as a co-treatment in a number of ANG II-mediated conditions. In particular, though not exclusively, the method will be beneficial in treatment of hypertension, hypertension related renal diseases or obesity.

Other aspects of the invention may become apparent from the following description, given by way of example and with reference to the experimental data.

### BRIEF DESCRIPTION OF THE FIGURES

This invention is described with reference to specific embodiments thereof. Other aspects and embodiments of this invention can be appreciated by reference to the detailed descriptions and figures, in which:

FIG. 1 depicts postnatal growth curves of AD (ad libitum fed) and UN (undernourished) offspring from weaning until commencement of IGF-I treatment (AD control diet (open circles), AD hypercaloric diet (filled triangles), UN control diet (open squares), UN hypercaloric diet (filled diamonds). n=6 per group, data are mean±SEM.

FIG. 2 depicts weight gain (grams per day) during 14 days of IGF-I treatment. Programming effect NS, IGF-I treatment effect p<0.0001, diet effect p<0.05, diet×IGF-I treatment interaction p<0.05. n=6 per group, data are mean±SEM.

FIG. 3 depicts food intake (kcal consumed per gram body weight per day) during 14 days of IGF-I treatment. Programming effect p<0.0005, IGF-I treatment effect p<0.0001, diet effect p<0.0001, programming×IGF-I treatment interaction p<0.005, programming×IGF-I treatment×diet interaction p<0.05. n=6 per group, data are mean±SEM.

FIG. 4 depicts change in systolic blood pressure after 14 days of IGF-I treatment. Programming effect p<0.0005, IGF-I effect p<0.005, diet effect NS. There were no significant statistical interactions. n=6 per group, data are mean±SEM.

FIG. 5 depicts blood plasma IGF-I concentrations. Programming effect NS, IGF-I treatment effect p<0.0001, diet effect NS, programming×IGF-I treatment interaction p<0.05. n=6 per group, data are mean±SEM.

FIG. 6 depicts fasting blood plasma insulin and glucose concentrations following 14 days IGF-I treatment. Insulin: programming effect p<0.05, IGF-I treatment effect p<0.0001, diet effect p<0.0005, diet×IGF-I treatment interaction p<0.0005. Glucose: programming effect NS, IGF-I treatment effect p<0.0001, diet effect p<0.0001. There were no significant statistical interactions for fasting plasma glucose concentrations. n=6 per group, data are mean±SEM.

FIG. 7 depicts retroperitoneal and gonadal fat pad weight (expressed as percent body weight), plasma leptin concentrations following 14 days saline (open bars) or IGF-I (closed bars) treatment and the relationship between adipose mass and plasma leptin concentrations. Retroperitoneal fat: programming effect p<0.05, IGF-I treatmecnt effect p<0.0001, diet effect p<0.0001. Gonadal fat: programming effect p<0.0001, IGF-I treatment effect p<0.0001, diet effect p<0.0001. Plasma leptin: programming effect p<0.005, IGF-I treatment effect p<0.0001, diet effect p<0.0005, programming×diet interaction p<0.05, diet×IGF-I interaction p<0.005. There were no significant statistical interactions for retroperitoneal and gonadal fat pad weight. n=6 per group, data are mean±SEM.

FIG. 8 depicts serum IGFBPs as quantified following ligand blotting analysis. IGFBP-3 (38-44 kDa): programming effect NS, IGF-I treatment effect p<0.0001, diet effect p<0.0001, programming×IGF-I treatment interaction p<0.0001, diet×IGF-I treatment interaction p<0.005, programming×IGF-I treatment×diet interaction p<0.05. IGFBP-1,-2 (28-30 kDa): programming effect NS, IGF-I treatment effect p<0.0001, diet effect p<0.05, programming×IGF-I treatment interaction p<0.05. IGFBP-4 (24 kDa): programming effect p<0.0001, IGF-I treatment effect p<0.0001, diet effect p<0.0005, programming×IGF-I treatment interaction p<0.005, diet×IGF-I treatment interaction p<0.05. 38 kDa IGFBP-3: programming effect p<0.0001, IGF-I treatment effect p<0.0001, diet effect p<0.0005. There were no significant statistical interactions for the 38 kDa IGFBP-3 band. Sample was 2 μl, n=6 per group, data are mean±SEM.

FIG. 9 depicts a photomicrograph of an immunohistochemical section of a programmed kidney incubated with the AT₁R antibody. Localisation of the AT₁R immunoreactivity (brown staining) can be seen distinctly in the medullary region (MR). Slight immunoreactivity is also evident in the cortical region (CTX). (Mag 100×).

FIG. 10 depicts a photomicrograph of the negative control immunohistochemical kidney incubated with normal rabbit serum. No evidence of AT₁R immunoreactivity was observed. (Mag×50).

FIG. 11 depicts photomicrographs of an immunohistochemical section of a programmed kidney incubated with the AT₁R. Renal cortex demonstrates labelling throughout the glomeruli (Glm) and renal tubules, specifically the proximal (PT) and distal (DT) tubules. (A: mag 250×, B: mag 1000×).

FIG. 12 depicts photomicrographs of an immunohistochemical section of a programmed kidney treated with IGF-1 incubated with the AT₁R. There is no evident labelling throughout glomeruli and renal tubules. (A: mag 250×, B: mag 1000×).

FIG. 13 depicts photomicrograph of an outer medullary immunohistochemical section of a programmed kidney incubated with the AT₁R. Distinct labelling can be seen in the renal tubules. (mag 250×)

FIG. 14 depicts a photomicrograph of the outer medullary immunohistochemical section of a programmed kidney treated with IGF-1 and incubated with the AT₁R. Decreased AT1R immunoreactivity is seen (mag 250×).

FIG. 15 depicts a photomicrograph of the outer medullary immunohistochemical section of a programmed kidney incubated with the AT₁R. Strong labelling of the proximal tubules is demonstrated with lesser staining within the distal tubules (mag 630×).

FIG. 16 depicts a photomicrograph of the outer medullary immunohistochemical section of a programmed kidney treated with IGF-1, incubated with the AT₁R. Little immunoreactivity is seen with both the proximal (T) and distal (DT) tubules (mag 630×).

FIG. 17 depicts histograms showing the localisation and intensity of the AT₁R in the programmed offspring. Values are expressed as mean×SEM.

### DETAILED DESCRIPTION

### Definitions

In general, the following words or phrases or abbreviations have the indicated definition when used in the description examples, and claims:

As used herein, “ACE” means angiotensin converting enzyme.

As used herein, “IGF-1 analogue” means a protein which is a variant of IGF-I through insertion, deletion or substitution of one or more amino acids, but which retains at least substantial functional equivalency.

As used herein, “ANG II” or “AII” means angiotensin II.

As used herein, “ANG II-like G protein-coupled seven transmembrane receptors” refer to any G protein-coupled receptor having seven transmembrane domains, similarly to angiontensin II receptors.

As used herein, “AT₁R” means angiotensin II type 1 receptor.

As used herein, “AT₂R” angiotensin II type 2 receptor.

As used herein, “angiotenisin II receptor” means a G protein-coupled receptor to which angiotensin II binds to and/or activates, or which angiotensin II is capable of activating and/or binding to.

As used herein, “G protein-coupled receptors” mean a cell surface receptor havaing seven transmembrane domains and are coupled to G-proteins (GTP (guanosine 5′-triphosphate)-binding protein). Many G-protein coupled receptors have seven membrane-spanning regions or domains and may be termed “G-protein seven transmembrane receptors.”

As used herein, “insulin-like growth factor” or “IGF-I” includes, IGF-I, a biologically active IGF-I analog, a biologically active IGF-I mimetic, a functionally equivalent ligand, a compound that increases the concentration of IGF-I, or a compound that increases the concentration of IGF-I analogs.

As used herein, “IGF-I” includes any mammalian insulin-like growth factor-I IGF-I, examples being porcine IGF-I, ovine IGF-I, equine IGF-I, bovine IGF-I or human IGF-1.

As used herein, the term “IGF-I” includes a full length native sequence or a variant form, and from any source, whether natural synthetic, or recombinant.

As used herein, “a biologically active IGF-I analog” means a protein which is a variant of IGF-I through insertion, deletion or substitution of one or more amino acids, but which retains at least substantial functional equivalency. IGF-I and analogs can be purified from natural sources or produced by recombinant DNA techniques.

For the purposes of the present invention “a biologically active IGF-I analog” includes any compounds which exert a biological effect similar to IGF-I and which include but are not limited to any naturally occurring active part of IGF-l (e.g. GPE or des(1-3) IGF-I), IGF-2, any naturally occurring active parts of IGF-2 (e.g. des(1-3)IGF-II) or any of their known synthetic analogs. Synthetic analogs of IGF-I include, but are not limited to LR3IGF-I, [Arg³]IGF-I, Long™R³IGF-I, [Ala³¹]IGF-I, Des(2,3)[Ala³¹]IGF-I, [Leu²⁴]IGF-I, Des(2,3)[Leu²⁴]IGF-I, [Leu⁶⁰]IGF-I, [Ala³¹][Leu⁶⁰]IGF-I, [Leu²⁴][Leu⁶⁰]IGF-I, etc.

The term “IGF-1 functionally equivalent ligand” means an agent that binds to and activates the receptors to which IGF-I binds to and activates to elicit an effect. In general, a protein is a functional equivalent of another protein for a specific function if the equivalent protein is immunologically cross-reactive with, and has at least substantially the same function as the original protein. The equivalent can be, for example, a fragment of the protein, a fusion of the protein with another protein or carrier, or a fusion of a fragment with additional amino acids. For example, it is possible to substitute amino acids in a sequence with equivalent amino acids using conventional techniques. Groups of amino acids normally held to be the equivalent are:

(a) Ala, Ser, Thr, Pro, Gly;

(b) Asn, Asp, Glu, Gln;

(c) His, Arg, Lys;

(d) Met, Leu, Ile, Val; and

(e) Phe, Tyr, Trp.

As used herein “a compound that increases the concentration of IGF-I, or a compound that increases the concentration of IGF-I analogs, or a compound that prevents inhibition of IGF-I activity” also includes one or more acid-labile subunits (ALS) of an IGF-I binding complex.

As used herein, “a compound that increases the concentration of IGF-I, or a compound that increases the concentration of IGF-I analogs, or a compound that prevents inhibition of IGF-I activity” includes compounds which maintain, store, transport or prolong half-life of the IGF-I in circulation, in particular IGF-I binding proteins, for example those currently known, i.e., IGF-BP1, IGFBP2, IGFBP3, IGFBP4, IGFBP5, IGFBP6, IGFBP7, IGFBP8.

The present invention also extends to the administration of a compound that either increases the concentration of IGF-I, increases the concentration of analogs of IGF-I or prevents inhibition of IGF-I activity. There is a wide variety of biological compounds that exert a stimulatory or inhibitory effect over IGF-1. Growth hormone, estrogen and thyroid hormone have all been reported to have stimulatory effects. There are also patents for design of IGF analogues (example U.S. Pat. No. 6,251,865) that inhibit binding to IGF binding proteins, and for IGF inhibitors (example U.S. Pat. No. 6,121,416) that prevent binding of IGF-1 to its receptor.

As used herein, “potential for signal transduction” or “signal transduction potential” refers to an ability of a receptor to interact in a cascade of processes to cause a change in the level of a second messenger, for example, calcium or cyclic AMP) that ultimately effects a change in cell's functioning.

As used herein, “hypertension” refers to persistently high arterial blood pressure. In humans this would normally equate to either a systolic pressure of greater than 140 mm Hg, a diastolic pressure of greater than 90 mm Hg.

As used herein, “hypertension related kidney diseases” refers to any renal pathology related to hypertension.

As used herein, “ACE inhibitor” refers to inhibitors of angiotensin converting enzyme, including drugs that exert haemodynamic effects by inhibiting production of ANG II. ACE inhibitors can result in vasodilation or mild natriuresis without directly affecting heart rate and/or myocardial contractility.

As used herein, “angiotensin II antagonist” refers to drugs that exert haemodynamic effects by blocking the binding of angiotensin II to the AT₁ receptor.

### Diagnosis and Characteristics of Fetal Programming

We have developed an animal model that demonstrates the significance of fetal programming on the subsequent development of metabolic disorders in adult life. This model involves “fetal programming” whereby under nutrition of the mother during gestation leads to development in the fetus, of a syndrome that includes hyperphagia, obesity, insulin resistance and hypertension in the offspring (hereafter referred to as “programming” or “programmed”). The model is described in more detail herein and substantially mimics a metabolic syndrome in humans known as “Syndrome X”. This animal model is well suited as a predictive tool, in that it closely mimics the human condition.

From this model it can be seen that many harmful conditions manifest in individuals due to the condition being brought about by a programming of the fetus to get those conditions later in life following birth. Once having noted this connection, treatment on a prophylactic basis and/or on a symptomatic basis can be proceeded with. Clearly, if a prophylactic treatment can be used that will lower or delay the incidence of the listed conditions in individuals that would be very advantageous. This connection between fetal programming and onset of any one or more of a number of harmful conditions represents a significant advance allowing treatment programs for those conditions to be developed.

Physical or metabolic indicators that can be used to identify an individual at risk include, but are not necessarily limited to, maternal food deprivation, placental dysfunction, uteroplacental blood supply problems, intrauterine growth retardation (usually as a result of the previously listed conditions) altered levels of IGF-1 and also evidence that the mother herself was exposed to “fetal programming” whilst in the womb (also known as “intergenerational effect”).

Children of lower birth weight can develop higher circulating concentrations of IGF-1 than expected for their height and weight. Undernutrition in utero can also lead to reprogramming of the IGF-1 axis. Increases in plasma IGF-1 concentrations in low birth weight children may be linked to post-natal catch-up growth. Hence, altered IGF-1 levels can be a useful indicator.

Exposure of the mother to fetal programming while in the womb can also be an predictor of fetal programming in her offspring producing an “intergenerational effect”. If a mother's development was affected, the health of her fetus may be also affected.

Assessment of individuals on the basis of any one or more of the above indicators can be used to form the basis for a treatment regime to treat the consequences of fetal programming as is discussed herein.

The effects of IGF therapy on programmed animals receiving an ordinary or a hypercaloric diet postnatally, were investigated. IGF-I treatment markedly reduced appetite, obesity, hyperinsulinemia, hyperleptinemia and hypertension in these programmed animals. Importantly, there was no significant effect on blood pressure in normotensive animals. The effects of IGF-I may involve restoration of a functional feedback between insulin and leptin and/or differential regulation of the insulin receptor substrate (IRS), renin-angiotensin system (RAS) and/or IGF-I receptor signalling pathways, perhaps via a differential effect on IGFBPs, although further work on this hypothesis is needed.

The effects of IGF-I shown in the animal model suggest that this substance also has value as a treatment for hyperinsulinemia or insulin resistance in subjects exposed to fetal programming, or indeed in those at risk of developing such conditions through fetal programming. In particular, it may have benefit in such subjects prior to the development of any outward or physiological symptoms.

Furthermore, IGF-I treatment may be of value in ameliorating or preventing the consequences of fetal programming in otherwise normal subjects. Such subjects would be selected according to their risk of developing hypertension, obesity, diabetes or other metabolic disorders as a consequence of exposure to particular conditions in utero (ie, through programming).

As a result the present invention provides a means of prophylactic treatment of the conditions resulting from fetal programming. Treatment may then continue to provide a therapeutic benefit for mammals showing symptoms of such conditions.

Subjects are selected for prophylactic treatment on the basis of a review of maternal history (as discussed above) during pregnancy. However, it will be appreciated that other indicators for selection of subjects for treatment may be identified, including particular metabolic indicators or a particular combination of metabolic indicators.

The early identification of programmed individuals who are otherwise normal healthy subjects, before they show any physiological signs of metabolic disturbance, enables effective management and prevention of the onset of hypertension, obesity, diabetes and other metabolic disorders; disorders which can be an enormous financial burden, with lifetime treatment. It may be that early treatment of such individuals will only delay onset of symptoms, however even such a delay is beneficial both in terms of an individual's quality of life and in terms of financial issues.

One application of the methods of the present invention is in humans, either as adults or juveniles, although the methods also can have application to non-human mammals.

### Regulation of Angiotensin II Receptors

Angiotensin II (ANG II) and angiotensin II binding receptors play a key role in the renin-angiotensin system (RAS) which is responsible for hormonal control of blood pressure and sodium and water homeostasis. Renin is an enzyme produced mainly in the renal juxtaglomerular apparatus, that cleaves the peptide angiotensinogen, normally present in the blood, kidney and other organs, to produce the peptide angiotensin I (ANG I). ANG I possesses almost no bioactivity and, upon action of angiotensin-converting enzyme (ACE), is cleaved to a bioactive peptide, ANG II. ANG II is a potent vasoconstrictor, which plays a major role in increasing blood pressure. Vasoconstrictive effects of ANG II are produced by its action on the non-striated smooth muscle cells, stimulation of the formation of the adrenergenic hormones epinephrine and norepinephrine as well as the increase of the activity of the sympathetic nervous system as a result of the formation of norepinephrine. In addition to this action, ANG II has proven to be active on the adrenal zona glomerulosa to induce aldosterone production and on the adrenal medulla and sympathetic nerve ends to promote catecholamine secretion. Additionally, ANG II stimulates vasopressin secretion and production of prostaglandins E2 and I2, and is involved in the glomerular filtering function and the renal uriniferous tubular sodium reabsorption mechanism. ANG II can increase renal plasma flow and glomerular filtration rate, effects that can promote urine formation. However, in diabetic rats, glomerular and proximal tubular ANG II receptors are less dense than in non-diabetic rats.

ANG II elicits its biological actions by binding to specific membrane bound receptors on target cells to activate multiple intracellular transduction pathways. ANG II acts at two major cellular receptors, angiotensin II type 1 receptor and angiotensin II type 2 receptor.

AT₁ receptor (AT₁R) and AT₂ receptor (AT₂R) belong to the class of G protein-coupled seven transmembrane receptors. AT₁R has been shown to mediate most of the traditionally recognized ANG II functions such as vasoconstriction, electrolyte homeostasis etc. There has been evidence of generally antagonistic actions between the ANG II receptor isoforms AT₁R and AT₂R in the pressor and depressor actions and the growth promotion and suppression (53, 63). ANG II receptors are present in a number of organs and systems including heart, kidney, gonad, and placenta; pituitary and adrenal glands; the peripheral vessels, adipose tissue and the central nervous system. In kidney the major sites expressing AT₁R are glomeruli, proximal tubules, vasculature and medullary interstitial cells.

### Insulin-Like Growth Factor-1 (IGF-1)

IGF-I has previously been shown to have vasodilatory effects and to improve cardiac function in healthy volunteers (49). IGF treatment has been associated with reduction of arteriolar resistance and an increase in capillary blood flow (50). Animal studies suggested a role for IGF-I as a mediator of cardiac hypertrophic responses (48). IGF-1 has been shown to improve renal function in normal kidneys as well as those suffering from acute and chronic renal failure (52).

The applicants have herein demonstrated that an effective amount of IGF-I is successful in ameliorating or preventing hypertension which is a consequences. of fetal programming. IGF-I treatment reduced systolic blood pressure (SBP) only in animals that were hypertensive as a result of fetal programning or postnatal hypercaloric nutrition, whereas systolic blood pressure in normotensive animals remained unaltered.

It has been speculated that IGF-I can interact with the RAS and may alter ANG II expression via AT₁ receptor regulation. However, the studies of IGF-I effect on AT₁R carried out so far have focused on IGF-I activity in myocyte renin-angiotensin system and the inhibitory effect of IGF-I overexpression on apoptosis. It has been shown that in myocytes overexpressing IGF-I, AT₁R protein was decreased further attenuating the response of myocytes to ANG II (58). It has been suggested that the down-regulation of angiotensinogen (AGT), renin and AT₁R on myocytes and the reduced synthesis and secretion of AT₂ in the presence of IGF-I may be critical in the mechanism of prevention of cell death by IGF-I (57) IGF-I was also found to interfere with the development of diabetic myopathy by attenuating the activation of AT₁R (54).

However, none of the previous studies have shown a direct effect of IGF-I treatment on the density, distribution and signal transduction potential of ANG II receptors; the applicants' invention is the first of this kind to utilize IGF-I administration to modulate ANG II receptors in mammalian kidney. Moreover, prior art literature taught that in vitro treatment of adrenal fasciculata-reticularis cells with IGF-I significantly increased AT₁R binding sites in those cells (56). Thus, it was not clear whether IGF-1 has any effects on ANG II receptor mediated phenomena, and whether IGF-1 has therapeutic application in treating disorders affecting the distribution and density of ANG II receptors.

Our unexpected findings point to insulin-like growth factor-I (IGF-I) as a new alternative therapy or a co-therapy in a number of ANG II-mediated conditions, in particular hypertension. It has been suggested that inhibition of the RAS by ACE inhibition or blockade of AT₁ receptors has a positive influence not only on hypertension but also brings about blood pressure (BP) independent renoprotective effects. (43). The selective ANG II receptor antagonism has been show to reduce insulin resistance and improve glucose tolerance. (51).

Administration of IGF-I not only achieves the benefits of selective ANG II receptor antagonism, including reduction of insulin resistance and improved glucose tolerance, but also has beneficial side effects not achieved by standard anti-hypertensive drugs. For example, blockade of age and fat mass regulated adipocyte angiotensin II receptors by IGF-I can prevent adipose tissue hypertrophy and can ameliorate obesity.

Recent literature has shown that IGF-I receptors can function as G protein-coupled receptors (47). Moreover, IGF-II receptors have been shown to interact with G proteins in a manner similar to that of conventional G receptor coupling, suggesting that a common G protein recognition mechanism is shared by IGF-II receptors and conventional G-coupled receptors (61). ANG II receptors belong to the class of G protein-coupled seven transmembrane receptors, which are representative of a larger receptor family.

The present invention comprises methods of administration of IGF-I compounds to modulate the density, distribution and the potential for signal transduction of the G protein-coupled receptor family.

The novel application of IGF-I disclosed in the invention provides the public with a beneficial alternative to the methods of blocking or inhibiting the action of RAS existing in the prior art. Moreover, the present invention provides a new method of enhancing the efficacy of the present methods of inhibiting ANG II activity.

### Methods for Regulating Angiotensin Receptors Using IGF-1

In general, IGF-I compounds of this invention can be directly administered to the mammal in therapeutically or prophylactically effective amounts by any suitable technique either singly, in combination with or in the presence of an ACE inhibitor or angiotensin antagonist.

An IGF-I compound may be administered orally or parenterally, in combination with one or more suitable carriers or excipients. An IGF-1 compound can be dissolved in sterile saline or water. In certain embodiments, an administration route is subcutaneous injection. Another series of embodiments include administration to the mammal of a replicable vehicle encoding the IGF-I, and IGF-1analogue or ligand. Such a vehicle (which may be a modified cell line or virus which expresses IGF-I/analogue/ligand within the mammal) has application in increasing the concentration of the active compound within the mammal for a prolonged period. Such a vehicle can form a part of an implant.

According to one aspect of the present invention methods are provided for ameliorating or preventing hypertensive consequences of fetal programming in an otherwise normal mammal, comprising administering to the mammal an effective amount of insulin-like growth factor (IGF-I), an analogue thereof, or a functionally equivalent ligand.

In other embodiments, the mammal exposed to fetal programming is identified from a review of maternal history during pregnancy.

In further embodiments, the fetal programming is identified by one or more physiological or metabolic indicators such as maternal food deprivation, placental dysfunction, uteroplacental blood supply, intrauterine growth retardation, altered levels of IGF-1, and inter-generational effects.

In other embodiments the IGF-1/ligand/analogue is encoded in a replicable vehicle.

In other aspects, the invention includes administration of IGF-I to modulate density, distribution and signal transduction of angiotesin II receptors in mammalian kidney.

The applicants have previously observed that administration of IGF-I reduces insulin resistance and improves glucose tolerance (Vickers et al. 2001), and thus, IGF-I administration achieves the beneficial effects of selective angiotensin II receptor antagonism comparable to those of angiotensin antagonists and ACE inhibitors. Moreover, the method disclosed in the present application has beneficial side effects not achieved by standard anti-hypertensive drugs. For example, modulation of age and fat mass regulated adipocyte angiotensin II receptors by IGF-I can prevent adipose tissue hypertrophy and ameliorate obesity.

The novel application of IGF-I disclosed in the present invention provides the public with a beneficial alternative to the methods of blocking or inhibiting the action of RAS known in the prior art. Moreover, the present invention describes a new method of enhancing the efficacy of the known methods of inhibiting ANG II activity.

In certain embodiments, IGF-1 modulated angiontensin II receptors or angiotensin II-like G protein-coupled seven transmembrane receptors are located in mammalian renal tissue, including in glomeruli; glomerular mesangial cells; inner stripe of the outer medulla; outer stripe of the outer medulla; inner medulla toward the tip of the papilla; proximal convoluted tubules; proximal tubular epithelia; vascular smooth muscle cells, in particular, efferent arteriolar vascular smooth muscle cells, and on luminal surface of proximal and distal tubule cells.

In other embodiments, the effective amount of an insulin-like growth factor-I (IGF-I) compound is administered in a form of a pharmaceutical composition including a pharmaceutically acceptable carrier thereof.

An effective amount of IGF-I compound can be administered by way of administration of a replicable vehicle encoding for IGF-I, a biologically active IGF-I analog, a biologically active IGF-I mimetic, a functionally equivalent ligand, a compound that increases the concentration of IGF-I, or a compound that increases the concentration of IGF-I analogs.

In certain embodiments, an effective amount of IGF-I compound is administered by intramuscular, subcutaneous or intraperintoneal injection or implant.

In yet further embodiments, the said effective amount of IGF-I compound is administered through intravenous, transdermal, transmucosal, oral, or epidural route.

To treat certain conditions, the effective amount of an insulin-like growth factor-I (IGF-I) compound is between 0.01 mg/kg/day and about 1 mg/kg/day.

A composition comprising an IGF-1 compound can be administered in a pharmaceutically acceptable combination with one or more suitable carriers or excipients.

A composition comprising an IGF-1 compound can be used for treatment, prophylaxis, attenuation of hypertension in the mammal.

In certain embodiments, a composition comprising an IGF-1 compound can be used for treatment, prophylaxis or attenuation of resulting from hypertension related kidney diseases in a mammal.

In other embodiments, a composition comprising an IGF-1 compound can be administered with at least one ACE inhibitor and/or angiotensin II antagonist.

In certain embodiments, an IGF-1 compound can be used along with one or more ACE inhibitors. Such ACE inhibitors include captopril, cilazapril, enalapril, fosinopril, imidapril, lisinopril, moexipril, perindopril, quinapril, ramipril, trandolapril and other known ACE inhibitors.

In other embodiments, an angiotensin II antagonist can be selected from a group that includes candesartan, irbesartan, losartan, telmisartan, valsartan or other known A II antagonists.

Further aspects the present invention comprises methods for enhancing the antihypertensive and renoprotective properties of ACE inhibitors and ANG II antagonists comprising the step of administering to a mammal an effective amount of an IGF-I compound, where an IGF-I compound comprises IGF-I, a biologically active IGF-I analog, a biologically active IGF-I mimetic, a functionally equivalent ligand, a compound that increases the concentration of IGF-I, or a compound that increases the concentration of IGF-I analogs in the presence of the said ACE inhibitor or the said angiotensin II antagonist.

### Therapeutic Administration of IGF-1

While methods of this invention can involve administering an effective amount of IGF-I, it may alternatively use an analogue thereof or a functionally equivalent ligand that will bind to the IGF-1 receptors. The IGF-I can be any mammalian IGF-I, with examples being human IGF-I, porcine IGF-I, or ovine IGF-I and bovine IGF-I. It is, however, preferred that the IGF-I used be human IGF-I where the mammal is a human. In addition to IGF-I itself, the use of analogues of IGF-I or functionally equivalent ligands is contemplated. It will be appreciated that these benefits may be derived from the administration of ligands which bind to the IGF-I receptor as well as to IGF-I itself.

By analogues of IGF-I is meant compounds that exert a similar biological effect to IGF-I and includes IGF-2 and analogues of IGF-2 naturally occurring analogues (eg. des(1-3) IGF-I) or any of the known synthetic analogues of IGF-I. IGF-I and analogues can be purified from natural sources or produced by recombinant DNA techniques. Recombinant IGF-I and des(1-3) IGF-I can be obtained commercially.

The present invention may also extend to the administration of an agent which either stimulates the production of IGF-I, or which lessens or prevents inhibition of IGF-I activity. There is a wide variety of biological compounds that exert a stimulatory or inhibitory effect over IGF-1. Growth hormone, estrogen and thyroid hormone have all been reported to have stimulatory effects. There are also patents for design of IGF analogues (example U.S. Pat. No. 6,251,865) that inhibit binding to IGF binding proteins, and for IGF inhibitors (example U.S. Pat. No. 6,121,416) that prevent binding of IGF-1 to its receptor. Such options could also be used in the present invention.

The active agent can be administered using any suitable route. Where IGF-I is the active agent, it may for example be administered orally or parenterally, in combination with one or more suitable carriers or excipients. Preferably the IGF-1 is dissolved in sterile saline or water. The preferred administration route is subcutaneous injection.

Another possibility is administration to the mammal of a replicable vehicle encoding the IGF-I/analogue/ligand. Such a vehicle (which may be a modified cell line or virus which expresses IGF-I/analogue/ligand within the mammal) could have application in increasing the concentration of the active compound within the mammal for a prolonged period. Such a vehicle could form part of an implant.

Methods for producing and using replicable vehicles are known in the art. Briefly, an oligonucleotide encoding the IGF-1 or analog thereof is inserted as an open reading frame operably linked to an initiation codon and a termination codon in a replicable vector that also has a promoter, optionally one or more enhancer regions. Additionally, such a replicable vehicle may contain one or more selectable markers to increase the efficiency of selecting a transformant that incorporates the desired replicable vehicle.

The invention also includes cells transformed with IGF-1 containing replicable vehicles. Examples of such cells include human and non-human cells. For example, to provide a source of IGF-1 to a human subject, it can be desirable to transform human cells with an IGF-1 containing vehicle. In some embodiments, it can be desirable to use autologous cells (i.e., cells from the subject to be treated). In other circumstances, heterologous cells can be transformed with the IGF-1 containing replicable vehicle. Methods for producing such replicable vehicles and transformed cells are known in the art (e.g., Sambrook and Russell, Molecular Cloning Third Edition, Cold Springs Harbor Press (2001), incorporated herein fully by reference).

Such transformed cells can then be implanted into a subject in need of treatment. After implantation, the transformed cells can express IGF-1 and release the IGF-1 into the subject being treated. In some embodiments, it can be desirable to inject such transformed cells into the site where the IGF-1 is needed.

The present invention can therefore be seen to provide the use of an effective amount of IGF-1/analogue/ligand in the manufacture of a medicament for prevention of the onset of conditions that may result from fetal programming. Such conditions have been discussed previously herein. The actual manufacturing methods can be those known to the skilled person. Recognition of the likelihood of symptom onset is an important feature.

Dosage levels will be formulation dependent due to volume load. The amount of IGF-1 that can be administered will depend on the method of delivery. However, a suitable dosage range of IGF-I or analogues formulated for injection may be in the range of 0.1 μg/kg/day to 1 mg/kg/day. Alternatively, a dosage rate can be from about 2 to 200 μg/kg/day, and in yet further embodiments, from about 10 μg/kg/day to about 100 μg/kg/day.

Dosages from 40 to 80 μg/kg/day, by subcutaneous injection once or twice daily, and continued for 2-5 years or more, may be appropriate in children or young adults.

All literature and patent citations are expressly incorporated herein fully by reference.

### EXAMPLES

The following examples are provided to illustrate certain features of the invention, but should not be construed as limiting the scope of the invention. All animal studies were approved by the Animal Ethics Committee of the University of Auckland.

### Example 1

### Development of Animals Subjected To Fetal Programming

### Materials and Methods

Virgin Wistar rats (age 100±5 days, n=15 per group) were time mated using a rat oestrous cycle monitor to assess the stage of oestrous of the animals prior to introducing the male. After confirmation of mating, rats were housed individually in standard rat cages containing wood shavings as bedding and free access to water. All rats were kept in the same room with a constant temperature maintained at 25° C. and a 12-h light:12-h darkness cycle. Animals were assigned to one of two nutritional groups: Group 1; undernutrition (30% of ad-libitum (UN)) of a standard diet throughout gestation, Group 2; standard diet (AD) throughout pregnancy. Food intake and maternal weights were recorded daily until birth. After birth, pups were weighed and litter size recorded. Pups from undernourished mothers were cross-fostered onto dams which received AD feeding throughout pregnancy. Litter size was adjusted to 8 pups per litter to assure adequate and standardised nutrition until weaning.

After weaning, female offspring from the two groups of dams a) AD offspring and b) offspring from undernourished mothers (UN) were divided into 2 balanced postnatal nutritional groups to be fed either a standard diet (total digestible energy 2959 kcal/kg, protein 19.4%, fat 5%, fat/energy ratio 15.21%, protein energy ratio 26.23) or a hypercaloric diet; (total digestible energy 4846 kcal/kg, protein 31.8%, fat 30%, fat/energy ratio 55.72%, protein/energy ratio 26.25%). The mineral and vitamin content in the two diets were identical and in accordance with the requirements for standard rat diets. The fat content of the hypercaloric diet is typical of that seen in many Western diets. Weights and food intake of all offspring were measured daily for the first 2 weeks then every second day. At day 175, systolic blood pressure measurements were recorded using tail cuff plethysmography. Rats were then weight matched and received either rh-IGF-I (3 μg/g/day) or saline by osmotic minipump (Model 2002, Alzet Corp, Palo Alto, Calif. US) for 14 days. On the day prior to sacrifice, a repeated systolic blood pressure was recorded. Rats were then fasted overnight and sacrificed by halothane anaesthesia followed by decapitation. Blood was collected into heparinised vacutainers and stored on ice until centrifugation and removal of supernatant for analysis All animal work was approved by the Animal Ethics Committee of the University of Auckland.

### Blood Pressure Measurements

Systolic blood pressure (SBP) was recorded by tail cuff plethysmography according to the manufacturers instructions (Blood pressure analyser IITC, Life Science, Woodland Hills, Calif., USA). Rats were restrained in a clear plastic tube in a heated room (25-28° C.). After the rats had acclimatised (10-15 min) the cuff was placed on the tail and inflated to 240 mmHg. Pulses were recorded during deflation at a rate of 3mmHg/sec and reappearance of a pulse was used to determine systolic blood pressure. A minimum of three clear SBP recordings were taken per animal and the coefficient of variation for repeated measurements was <5%.

### IGF-I Infusion

At day 175, rats were weight matched (n=6 per group) and received either rh-IGF-I (Genentech Code #G117AZ, Batch c9831AY) or saline by osmotic minipump (Model 2002, Alzet Corp, Palo Alto, Calif. US). The dose was 3 μg/g/day for 14 days with a pump delivery rate of 5 μl per hour. The mean pump rate for the batch (Lot # 167258) of pumps used was 5.23±0.2 μl/hr. Pumps containing the IGF-I or saline solution were incubated in sterile saline for 4 hours at 37° C. prior to implantation. The osmotic pumps were implanted subcutaneously, under halothane anesthesia, using a small incision made in the skin between the scapulae. Using a haemostat, a small pocket was formed by spreading apart the subcutaneous connective tissues. The pump was inserted into the pocket with the flow moderator pointing away from the incision. The skin incision was then closed with sutures. All animals (n=48) were housed individually for the duration of the study.

### Radioimmunoassay (RIA) for Rat Insulin-Like Growth Factor-I (IGF-I)

IGF-I in rat blood plasma was measured using a IGF binding protein (IGFBP) blocked RIA described previously (22). The half maximally effective dose, or ED-50, was 0.1 ng/tube and the intra- and inter-assay coefficients of variation were <5% and <10% respectively.

### RIA for Rat Insulin

Rat insulin was measured by RIA as described previously (3). Blood plasma was diluted 1:4 in assay buffer (0.01M PBS containing 0.37% Na EDTA and 0.5% BSA, pH 6.2). In brief, the primary antibody, (guinea-pig anti-ovine-Insulin) was diluted in assay buffer to an initial working dilution of 1:80000. 0.1 ml of diluted sa1mple, control, or standard (rat insulin, 0.01-10 ng/ml, Crystal Chem., Chicago) was incubated with 0.2 ml of primary antibody for 24 hours at room temperature. 0.2 ml ¹²⁵I-rh-Insulin (Eli Lily, Lot No 615-707-208) was then added at 15-20000 counts per tube. Equilibrium conditions were established after 24 hours incubation at 4° C. A second antibody was used to separate bound from free ligand as outlined previously (23) and the pellet counted by gamma counter. Rat plasma samples showed parallel displacement to the standard curve and recovery of unlabelled rat insulin was 96.5±4.4% (mean±SEM, n=11). The half-maximally effective dose (ED-50) was 0.5 ng/ml.

### RIA for Rat Leptin

A double antibody RIA was developed and validated for measurement of leptin in rat plasma. An antibody was raised in rabbits against a fragment (aa 30-45) of bovine leptin. Standard preparation was rm-leptin (Crystal Chem, US., #CR-6781) used in concentrations ranging from 0.5 to 20 ng/ml. Samples were assayed neat or diluted 1:2-1:4 in assay buffer (0.05M PBS, pH 7.4 containing 0.1M NaCl, 0.5% BSA, 10 mM EDTA, 0.05% NaN₃). In brief, 100 μl of primary antibody (1:25000) was added to tubes containing 100 ul of sample or standard. Following incubation for 24h at 4° C., 100 μl of tracer (¹²⁵I-rm-leptin, 20000 cpm per tube) was added to all tubes followed by a further incubation for 24 h at 4° C. A second antibody technique to separate bound from free ligand was used as outlined previously (23). Rat plasma samples showed parallel displacement to the standard curve and recovery of unlabelled rn-leptin was 101.4±2.7% (mean±SEM, n=26). The ED-50 was 0.37 ng/ml and the intra-assay coefficient of variation was <5% (all samples measured within a single assay).

### Blood Biochemistry

Plasma glucose concentrations were measured using a YSI Glucose Analyzer (Model 2300, Yellow Springs Instrument Co., Yellow Springs, Ohio, US). Blood plasma free fatty acids were measured by diagnostic kit (Boehringer-Mannheim #1383175). All other plasma analytes were measured by a BM/Hitachi 737 analyser by Auckland Healthcare Laboratory Services.

### Ligand Blotting of Rat Plasma IGFBPs

IGFBPs in rat plasma (2 μl sample, n=6 per treatment group) were analyzed by ligand blotting (24) as described in detail elsewhere (25). Rat ¹²⁵I-IGF-II was used as radiolabel. Nitrocellulose blots were air dried and exposed to Kodak X-Omat AR diagnostic film (Eastman Kodak, Rochester, N.Y., USA) in Amersham Hyperscreen cassettes with intensifier screens. For quantification, nitrocellulose blots were exposed overnight to phospor imaging screens and analysed on a Storm Phosporlmager system using ImageQuant software (Molecular Dynamics, Sky Valley, Calif., USA). All values were expressed relative to a normal rat plasma pool and standardised to 100% for control group. The IGFBPs were identified on the basis of their molecular size using nomenclature previously described (26).

### Statistical Analysis

Statistical analyses were carried out using SigmaStat™ (Jandel Scientific, San Rafael, Calif., USA) and StatView™ (SAS Institute Inc., NS, USA) statistical packages. Differences between groups were determined by two-way (pre-IGF-I treatment) or three-way ANOVA (post-IGF-I treatment) followed by Bonferonni post-hoc analysis and data are shown as mean±SEM. Plasma leptin and food intake data were also analysed by ANCOVA using unadjusted fat pad weight and body weights as covariates respectively. Statistical significance was assumed at the p<0.05 level.

### Results

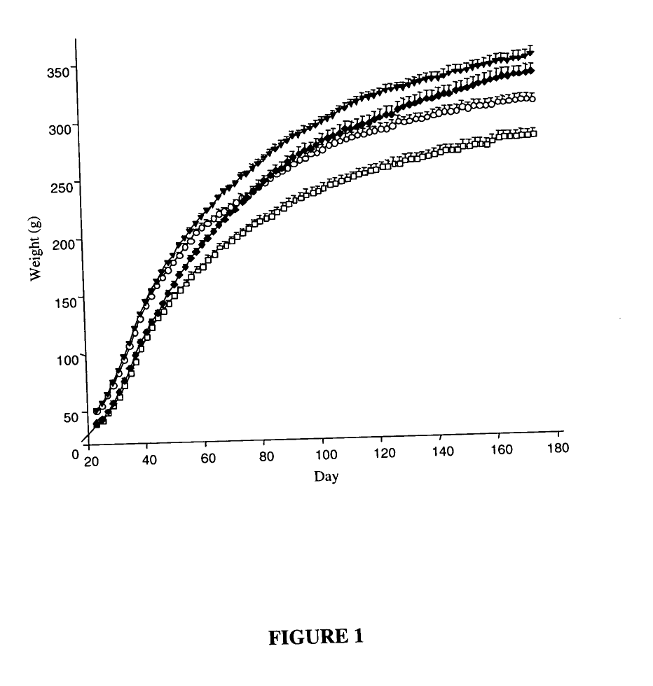

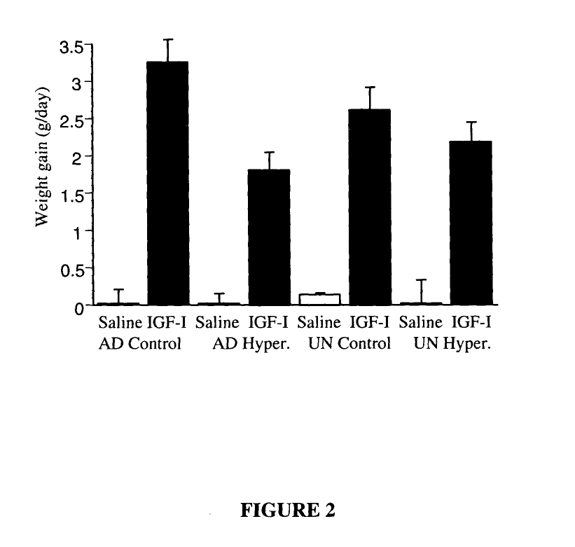

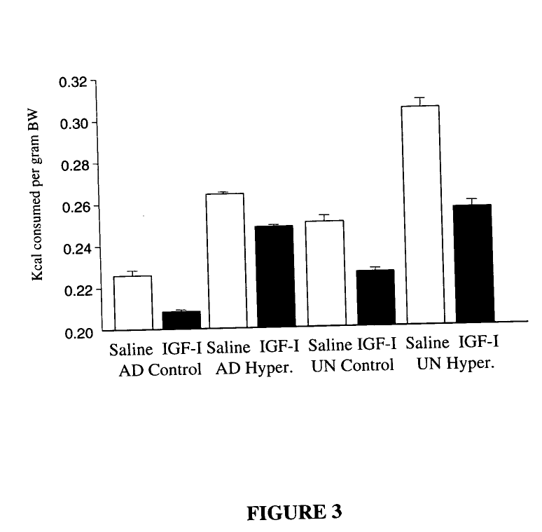

Maternal undernutrition resulted in fetal growth retardation reflected by significantly decreased body weight at birth in the offspring from UN dams (UN 4.02±0.03 g, AD 6.13±0.04 g, p<0.001). Litter size was not significantly different between the two groups (AD 11.7±1.93, UN 11.2±2.03). From birth until weaning at day 22, body weights remained significantly lower in the UN offspring (AD 51.5±0.6 g,UN 37.8±0.9 g). Total body weights on each diet remained significantly lower (p<0.0001) in UN offspring for the remainder of the study. Hypercaloric nutrition during postnatal life resulted in significantly (p<0.0001) increased body weights compared to control fed animals and by postnatal day 100 UN animals fed hypercalorically showed apparent catch-up growth to match the body weight of AD animals fed the control diet (FIG. 1). Body weight gain was increased in all IGF-I treated animals (FIG. 2) and no difference in growth response was observed between AD and UN offspring. However, daily weight gain was significantly reduced in animals treated with IGF-I on hypercaloric nutrition as reflected by the significant (p<0.05) diet×IGF-I interaction. UN offspring were shorter than AD offspring in each treatment group and nose-anus lengths were significantly (p<0.05) increased in all IGF-I treated animals (Table 2). UN animals showed a significantly higher food intake on both diets compared to AD animals. Food intake was reduced (p<0.005) in all IGF-I treated offspring (FIG. 3). A significant statistical interaction was observed between programming and IGF-I treatment whereby reduction in food intake was more pronounced in UN animals following IGF-I treatment (p<0.005).

Prior to onset of IGF-I therapy, SBP was markedly elevated (p<0.000l) in UN offspring on the control diet compared to AD offspring. The programming effect on hypertension was markedly amplified by postnatal exposure to hypercaloric nutrition (Table 1). SBP was significantly reduced with IGF-I therapy in UN animals and in the group of AD offspring which had elevated blood pressure as a result of postnatal hypercaloric nutrition (FIG. 3).

TABLE 1
Systolic blood pressure (SBP)(mmHg) prior to onset of IGF-I therapy.

| AD Control | UN control | AD hypercaloric | UN hypercaloric |
|---|---|---|---|
| (mmHg) | (mmHg) | (mmHg) | (mmHg) |
| 121.84 ± 1.67 | 140.47 ± 2.122 | 140.04 ± 2.63 | 148.43 ± 1.59 |

Data were analysed by two-way ANOVA. Data is mean±SEM with n=12 animals per group. There were no significant statistical interactions.

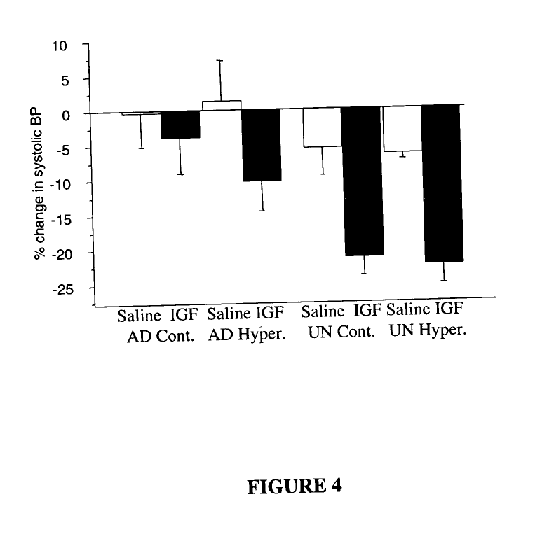

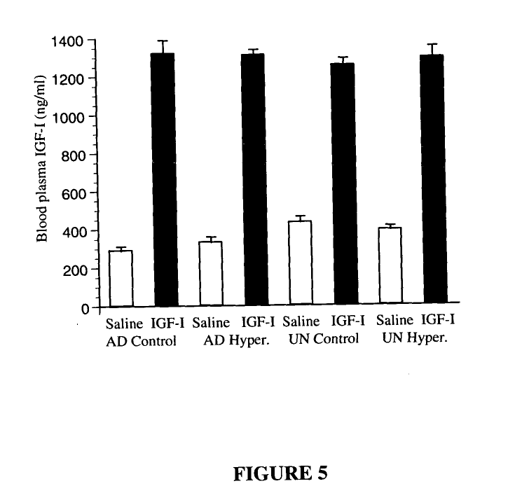

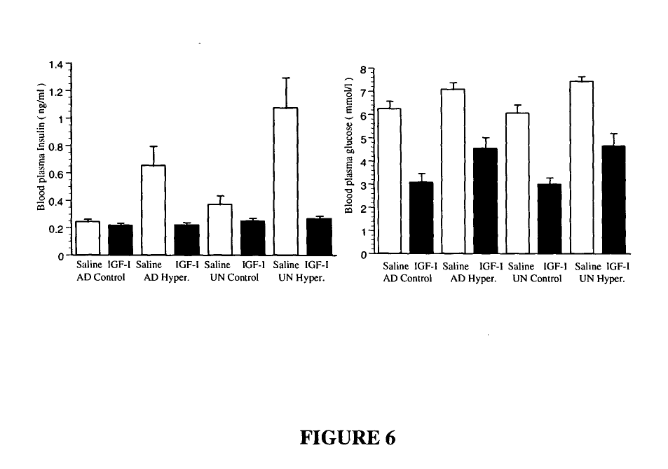

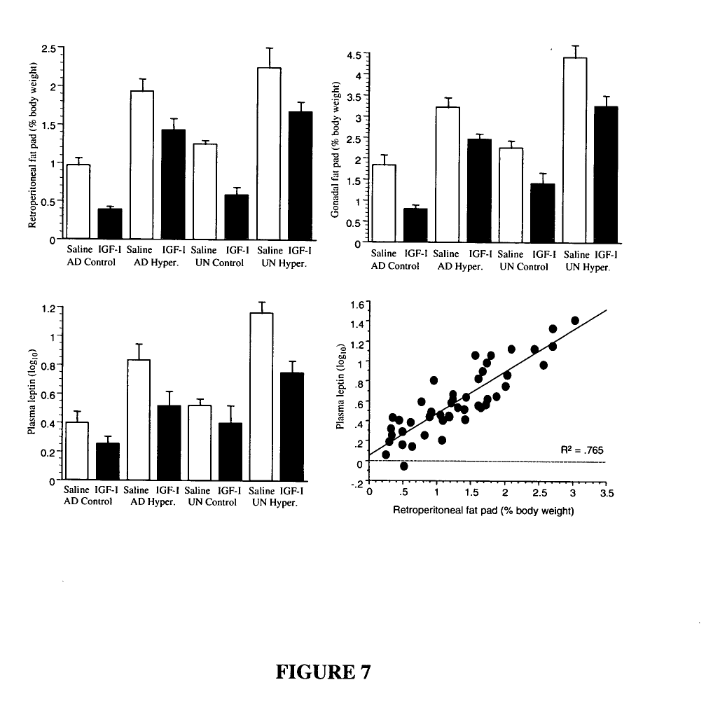

Blood plasma IGF-I concentrations were markedly increased (p<0.0001) in all IGF-treated offspring (FIG. 4). The rise in plasma IGF-I concentrations following IGF-I treatment was less in UN animals on both diets compared to AD animals (programming/diet interaction p<0.05). Fasting plasma insulin levels were higher (p<0.05) in UN offspring and were further elevated by hypercaloric nutrition (p<0.0005). Treatment with IGF-I significantly lowered insulin concentrations (p<0.005) in all offspring (FIG. 6); this effect was most marked in the animals on hypercaloric nutrition (IGF-I treatment x diet interaction p<0.005, FIG. 5). Plasma glucose was not different between AD and UN offspring but was increased (p<0.0001) by hypercaloric nutrition (FIG. 6). IGF-I treated animals showed markedly reduced plasma glucose concentrations (p<0.0001)(Figure 5). Plasma leptin concentrations were higher (p<0.005) in UN offspring and were increased (p<0.000l) by hypercaloric diet. IGF-I treatment significantly lowered plasma leptin concentrations (p<0.0005). As observed with insulin, there was a strong diet-IGF-I treatment interaction (p<0.005, FIG. 7) with plasma leptin levels being most markedly reduced in offspring fed hypercalorically. Regression analysis revealed a strong positive relationship between plasma leptin and fasting insulin concentrations (r²=0.75, p<0.000l). Retroperitoneal and gonadal fat pads were significantly larger in UN offspring (p<0.05) and were further increased by hypercaloric nutrition in both AD and UN offspring (p<0.000l). Treatment with IGF-I significantly reduced fat pad mass in all treated animals (p<0.0001, FIG. 7). Regression analysis showed a highly significant positive relationship between fat mass and fasting plasma leptin (r²=0.78, p<0.001).

TABLE 2
Body weight, length and tissue weights of AD and UN offspring (age 190 ± 5 days)
following 14 days treatment with IGF-I.

| | | Body | | | | | | Nose-Anus |
|---|---|---|---|---|---|---|---|---|
| Group | Tx | weight (g) | Heart | Liver | Kidney | Spleen | Adrenal | (mm) |
| AD Control diet | CBS | 284 ± 5.3 | 0.38 ± 0.01 | 2.70 ± 0.07 | 0.80 ± 0.03 | 0.24 ± 0.01 | 0.028 ± 0.001 | 209 ± 2.3 |
| | IGF-I | 322 ± 3.8 | 0.40 ± 0 01 | 2.63 ± 0.03 | 0.93 ± 0.02 | 0.38 ± 0.01 | 0.036 ± 0.002 | 212 ± 2.1 |
| AD Hypercaloric | CBS | 341 ± 6.7 | 0.35 ± 0.01 | 2.66 ± 0.10 | 0.74 ± 0.02 | 0.21 ± 0.01 | 0.025 ± 0.001 | 208 ± 2.6 |
| diet | IGF-I | 374 ± 9.6 | 0.37 ± 0.01 | 2.50 ± 0.06 | 0.83 ± 0.02 | 0.38 ± 0.01 | 0.030 ± 0.002 | 217 ± 1.7 |
| UN Control diet | CBS | 258 ± 6.1 | 0.38 ± 0.01 | 2.57 ± 0.06 | 0.76 ± 0.02 | 0.25 ± 0.02 | 0.028 ± 0.001 | 195 ± 3.7 |
| | IGF-I | 297 ± 6.7 | 0.41 ± 0.01 | 2.50 ± 0.04 | 0.83 ± 0.03 | 0.36 ± 0.02 | 0.033 ± 0.001 | 203 ± 2.8 |
| UN Hypercaloric | CBS | 339 ± 8.9 | 0.33 ± 0.01 | 2.72 ± 0.04 | 0.68 ± 0.02 | 0.21 ± 0.01 | 0.025 ± 0.001 | 203 ± 1.7 |
| diet | IGF-I | 375 ± 11.4 | 0.36 ± 0.02 | 2.45 ± 0.04 | 0.73 ± 0.03 | 0.40 ± 0.05 | 0.028 ± 0.002 | 212 ± 4.7 |
| Programming effect | | p < 0.05 | NS | NS | p < 0.0001 | NS | NS | p < 0.0005 |
| IGF-I effect | | p < 0.0001 | p < 0.05 | p < 0.005 | p < 0.0001 | p < 0.0001 | p < 0.0001 | p < 0.05 |
| Diet effect | | p < 0.0001 | p < 0.0001 | NS | p < 0.0001 | NS | p < 0.0005 | p < 0.005 |
| Interactions | | | | | | | | |
| Programming × Diet | | p < 0.05 | NS | NS | NS | NS | NS | NS |
| Programming × IGF-I | | NS | NS | NS | NS | NS | NS | NS |
| Diet × IGF-I | | NS | NS | NS | NS | NS | NS | NS |
| Programming × | | NS | NS | NS | NS | NS | NS | NS |
| diet × IGF-I | | | | | | | | |

Data analysed by three-way factorial ANOVA followed by Bonferroni comparison. n = 6 animals per group, data are mean ± SEM.

Kidney weight was significantly (p<0.000l) reduced in UN offspring (Table 2). AD and UN offspring fed hypercalorically had relatively lighter kidneys (p<0.000l). Treatment with IGF-I significantly increased kidney weight (p<0.000l). Heart weight was not different between AD and UN offspring but was reduced relative to body weight in animals fed hypercaloric nutrition. IGF-I treatment caused an increase in heart weight in all treated animals (p<0.05). Liver weight was not different between AD and UN offspring and were not affected by diet. IGF-I treated animals had lighter livers relative to body weight compared to saline controls (p<0.005). Spleen weight was not different between AD and UN offspring and was not altered by diet. However, treatment with IGF-I caused a significant increase in spleen weight in AD and UN treated animals (p<0.000l). Relative brain weight in UN offspring was reduced as compared to AD offspring and was lighter relative to body weight (p<0.0001) in animals fed hypercalorically and/or treated with IGF-I. Adrenal weight was not different between UN and AD animals but was significantly (p<0.000l) increased with IGF-I treatment (Table 2).

Plasma free fatty acid concentrations were reduced in hypercalorically fed animals (p<0.005, Table 3) but there was no effect of programming or IGF-I treatment. Plasma urea concentrations were markedly lower in UN offspring (p<0.05, Table 3) and were decreased in all hypercalorically fed offspring (p<0.000l). Treatment with IGF-I caused a significant reduction (p<0.000l) in urea concentrations in all treated offspring. Plasma creatinine levels were not different between AD and UN offspring and were unaffected by diet. Treatment with IGF-I lowered (p<0.000 1) creatinine concentrations in all treated animals (Table 3).

TABLE 3
BLOOD BIOCHEMISTRY OF AD AND UN OFFSPRING (AGE 190 ± 5 DAYS)
FOLLOWING TREATMENT WITH IGF-I.

| | | Urea | Creatinine | Albumin | Magnesium | Calcium | Potassium | FFA | ALT | Bilirubin |
|---|---|---|---|---|---|---|---|---|---|---|
| Group | Tx | mmol/l | mmol/l | g/l | mmol/l | mmol/l | mmol/l | mmol/l | mmol/l | mmol/l |
| AD | CBS | 6.42 ± 0.12 | 0.058 ± 0.003 | 31.0 ± 1.03 | 0.815 ± 0.02 | 2.63 ± 0.03 | 6.83 ± 0.05 | 1.169 ± 0.13 | 30.7 ± 2.1 | 5.67 ± 0.5 |
| Control diet | IGF-I | 4.27 ± 0.25 | 0.047 ± 0.002 | 29.6 ± 0.49 | 0.945 ± 0.03 | 2.64 ± 0.04 | 7.02 ± 0.29 | 1.118 ± 0.13 | 54.2 ± 7.6 | 7.33 ± 0.4 |
| AD | CBS | 5.12 ± 0.35 | 0.057 ± 0.002 | 33.3 ± 0.42 | 0.898 ± 0.01 | 2.65 ± 0.04 | 6.88 ± 0.18 | 0.923 ± 0.06 | 35.6 ± 3.6 | 7.33 ± 0.6 |
| Hypercaloric | IGF-I | 3.02 ± 0.21 | 0.050 ± 0.002 | 29.6 ± 1.20 | 0.996 ± 0.04 | 2.61 ± 0.03 | 6.38 ± 0.30 | 0.945 ± 0.08 | 51.8 ± 2.1 | 7.40 ± 1.2 |
| UN | CBS | 5.88 ± 0.42 | 0.057 ± 0.002 | 28.0 ± 0.52 | 0.855 ± 0.01 | 2.67 ± 0.03 | 6.75 ± 0.45 | 1.013 ± 0.13 | 39.6 ± 7.6 | 7.30 ± 0.7 |
| Control diet | IGF-I | 3.72 ± 0.29 | 0.050 ± 0.001 | 31.0 ± 1.06 | 1.01 ± 0.04 | 2.81 ± 0.08 | 7.45 ± 0.21 | 1.187 ± 0.16 | 55.8 ± 3.5 | 7.67 ± 1.4 |
| UN | CBS | 4.53 ± 0.12 | 0.055 ± 0.002 | 30.2 ± 0.60 | 0.867 ± 0.01 | 2.70 ± 0.02 | 6.40 ± 0.19 | 0.805 ± 0.07 | 34.6 ± 0.8 | 5.83 ± |
| Hypercaloric | | | | | | | | | | 0.47 |
| | IGF-I | 3.03 ± 0.26 | 0.049 ± 0.001 | 28.2 ± 0.60 | 0.92 ± 0.02 | 2.68 ± 0.02 | 6.35 ± 0.36 | 0.889 ± 0.04 | 45.8 ± 3.0 | 5.83 ± 0.6 |
| Pro- | | P < 0.05 | NS | p < 0.05 | NS | p < 0.05 | NS | NS | NS | NS |
| gramming | | | | | | | | | | |
| effect | | | | | | | | | | |
| IGF-I effect | | p < 0.0001 | p < 0.0001 | NS | p < 0.0001 | NS | NS | NS | p < 0.0001 | NS |
| Diet effect | | p < 0.0001 | NS | NS | NS | NS | p < 0.05 | p < 0.005 | NS | NS |
| Interactions | | | | | | | | | | |
| Pro- | | NS | NS | NS | P < 0.05 | NS | NS | NS | NS | p < 0.05 |
| gramming × | | | | | | | | | | |
| Diet | | | | | | | | | | |
| Pro- | | NS | NS | p < 0.05 | NS | NS | NS | NS | NS | NS |
| gramming × | | | | | | | | | | |
| IGF-I | | | | | | | | | | |
| Diet × IGF-I | | NS | NS | p < 0.005 | NS | NS | NS | NS | NS | NS |
| Pro- | | NS | NS | NS | NS | NS | NS | NS | NS | NS |
| gramming × | | | | | | | | | | |
| diet × IGF-I | | | | | | | | | | |

Alanine aminotransferase (ALT) concentrations were significantly increased (p<0.0001) in IGF-I treated offspring but were not different between AD or UN offspring and were unaltered by hypercaloric nutrition (Table 3). Albumin concentrations were significantly (p<0.05) lower in UN offspring but there was no effect of diet or treatment. Calcium levels were higher (p<0.05) in UN offspring but there was no effect of diet or treatment. Plasma magnesium concentrations were markedly increased (p<0.0001) with IGF-I treatment but were unaffected by diet and were not different between AD and UN offspring (Table 3).

Plasma IGFBPs were analysed using nomenclature previously described (1,26). The 38-44 kDa, 28-30 kDa and 24 kDa bands represent IGFBP-3, IGFBP-1,-2 and IGFBP-4 respectively.

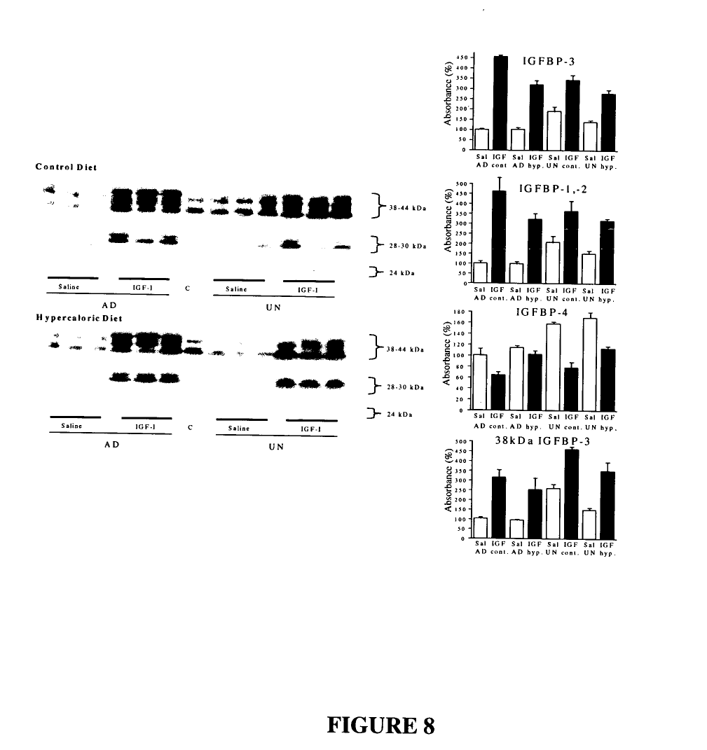

Analysis of plasma IGFBPs revealed that basal levels of the different IGFBPs were elevated in UN offspring compared to AD offspring. IGF-I treatment resulted in a 3- to 5-fold increase (p<0.001) in IGFBP-3 in all IGF-I treated animals (FIG. 8). However, there was a diminished up-regulation of IGFBP-3 in UN animals indicated by a significant (p<0.0001) programming×IGF-I treatment interaction (p<0.0001). Hypercaloric nutrition significantly (p<0.0001) reduced the IGFBP-3 band compared to animals on the control diet and diminished (p<0.0001) the up-regulation of IGFBP-3 following IGF-I treatment which was further amplified in UN animals with a significant (p<0.05) programming×diet×IGF-I treatment interaction. Interestingly although in UN animals the combined 38-44 kDa IGFBP-3 band showed impaired up-regulation following IGF-I treatment, analysis of the 38 kDa band alone showed a marked increase in this band in UN animals indicating a differential pattern of up-regulation in UN animals.

Treatment with IGF-I significantly (p<0.0001) increased (2 to 5 fold) the 28-30 kDa bands representing IGFBP-1 and 2 and as observed with IGFBP-3 there was a diminished up-regulation of IGFBP-3 following IGF-I treatment in UN animals compared to AD animals (p<0.05). Similarly, hypercaloric nutrition significantly reduced the increase in IGFBP-1 and 2 following IGF-I treatment.

The 24kDa band representing IGFBP-4 was significantly elevated in all UN animals (p<0.000l) and was further amplified in all animals fed hypercalorically (p<0.000l). In an opposing pattern to what was observed with IGFBP-1 to -3, a significant (p<0.000l) down-regulation of IGFBP-4 was observed following IGF-I treatment. A significant (p<0.00l) programming x IGF-I treatment interaction revealed that IGFBP-4 was more markedly down-regulated in UN animals following IGF-I treatment compared to AD animals. A significant diet x IGF-I treatment interaction was observed with IGF-I treatment resulting in a lesser reduction in IGFBP-4 in hypercalorically fed animals compared to those fed the control diet.

Thus, IGF-I treatment leads to a significant increase in body length, a marked reduction in food intake, decreased body fat mass and normalisation of blood pressure. Further endocrine responses include normalisation of fasting insulin and glucose concentrations and a major reduction in plasma leptin concentrations. The observation of a reduction in food intake despite the plasma leptin and insulin lowering effects of IGF-I invites a novel interpretation of IGF-I action. Firstly, IGF-I treatment may abolish the programming-induced leptin resistance at the leptin-hypothalamic circuitry and at the pancreatic adipoinsular feedback system. Secondly, IGF-I treatment may also ameliorate insulin resistance, both centrally and peripherally.

UN animals were hyperphagic on both postnatal diets compared to AD animals confirming earlier observations (3). However, the significant decrease in plasma leptin concentrations following IGF-I treatment was associated with a decrease in food intake. The decrease was more pronounced in offspring that were programmed to become obese and hyperphagic in adult life and may explain the reduced body weight gain observed in IGF-I treated offspring fed hypercaloric nutrition. The reduced food intake following IGF-I treatment may be the result of the anorectic effect of IGF-I via its insulin-sensitizing effects and reduction of chronic hyperinsulinemia. Food intake was most markedly reduced in programmed animals fed hypercaloric nutrition; the same animals that showed the most marked decrease and normalisation of fasting insulin concentrations and normoglycemia following IGF-I treatment.

Our data on the lipolytic effect of IGF-I support that of others (27-29) and suggest that the effects of prolonged IGF-I treatment on adipose tissue are not insulin-like as reflected by increased lipolysis and decreased body fat mass. IGF-I treatment may reduce body fat mass via an inhibition of the lipogenic capacity of adipocytes and reduction of lipogenesis in adipose tissue via inhibition of insulin secretion. IGF-I showed marked lipolytic effects with retroperitoneal and gonadal fat pad mass being markedly reduced concomitant with a significant decrease in fasting plasma leptin concentrations.

An endocrine feedback loop between insulin and leptin, the adipoinsular axis, has recently been proposed (30) and it has further been suggested that conditions of increasing adiposity and prolonged elevation of plasma leptin concentration result in a dysregulation of the adipoinsular axis (31,32). Our data add support to this concept and suggest that the interaction between the leptin and insulin signalling networks is disrupted as a result of fetal programming and further exacerbated by postnatal hypercaloric nutrition. Such a dysregulation of the adipoinsular axis may contribute to some of the alterations in the effects of insulin action that are involved in the progression to insulin resistance and adipogenic diabetes. Insulin receptor substrates 1 and 2 (IRS-1 and IRS-2) co-ordinate essential effects of insulin/IGF upon peripheral metabolism and beta cell function. Recent evidence suggests that impaired IRS-1 expression and downstream signalling events in adipocytes in response to insulin are associated with insulin resistance and the pentad of hypertension, hyperinsulinemia, dyslipidemia, obesity, and cardiovascular disease, known as Syndrome X (33). Furthermore, chronic hyperinsulinemia downregulates the mRNA for IRS-2, an essential component of the hepatic insulin signalling pathway, thereby exacerbating the insulin resistant state (34). Leptin can modify insulin-induced changes in gene expression in vivo (35) and the high concentrations of leptin required to obtain inhibition of signal transduction reflect the hyperleptinemia associated with obesity in the insulin resistant state (36). Furthermore, IRS-2 plays a special role in carbohydrate metabolism through mediation of both peripheral insulin action and pancreatic beta cell function. Pancreatic beta cells express little or no insulin receptor but large amounts of type 1 IGF-1 receptor which are proposed to promote islet and beta cell growth and survival, especially to compensate for peripheral insulin resistance (37). IGF-I has been shown to inhibit insulin secretion from beta cells through an IGF-1 receptor-mediated pathway (38,39) and the IGF-I-IRS-2 signalling pathway has been proposed to be critical for postnatal beta cell function (37). On the basis of this information and our results, treatment with IGF-I may restore some of the cross talk between leptin and insulin via a differential modification of the metabolic and mitotic effects of insulin exerted through IRS-1 and IRS-2 and the downstream signalling events they activate.

The highly significant increase in kidney weight with IGF-I treatment may be an important factor in the reduction of systolic blood pressure via changes in renal plasma flow and glomerular filtration rate. IGF-I treatment may reduce blood pressure by down-regulating the local renin-angiotensin system (RAS). Importantly, IGF-I treatment only reduced systolic blood pressure in animals that were hypertensive as a result of fetal programming and postnatal hypercaloric nutrition, while systolic blood pressure in normotensive animals remained unaltered.

This effect on blood pressure may result from improved insulin sensitivity and glycemic control in conjunction with the known vasodilatory effects of exogenous IGF-I. It may also, or alternatively involve the effects of IGF-I on leptin, which is believed to play an important role in the pathogenesis of obesity-related hypertension (40,41).

The effect of IGF-I treatment on improving insulin sensitivity and ameliorating the postnatal pathophysiology following fetal programming may be mediated by circulating IGFBPs. As IGF actions are modified by IGFBPs, the induction of binding proteins by IGF-I may act as a regulator of IGF-I in target tissues. We investigated the circulating levels of IGFBPs to examine whether fetal programming results in a differential expression of IGFBPs and whether such expression is altered by postnatal hypercaloric nutrition. The mechanism underlying the preferential up-regulation of the 38 kDa IGFBP-3 band in UN animals following IGF-I treatment is unclear.

Fetal programming resulted in a significant elevation in IGFBP-4 concentrations which were markedly amplified by postnatal hypercaloric nutrition. Treatment with IGF-I resulted in a significant decrease in circulating IGFBP-4 in all treated animals and, moreover, IGF-I treatment was more effective in reducing IGFBP-4 concentrations in those animals that had become obese as a result of fetal programming and hypercaloric nutrition. Activation of IGFBP-4 proteases by exogenous IGF-I may result in the degradation and inactivation of IGFBP-4. Our data show a reduced up-regulation of IGFBP-3 with IGF-I treatment following fetal programming concomitant with a more pronounced decrease in serum IGFBP-4 concentrations. Thus, IGFBP-4 induced restraint on IGF-I activity at the tissue level may be reduced and could partially explain the amelioration of programming and diet- induced pathophysiology observed. To date, these data are the first to report an impaired and differential up-regulation of IGFBPs following IGF-I treatment in adults which have been subjected to fetal programming; an impairment which is significantly altered by exposure to hypercaloric nutrition postnatally.

### Discussion

As we have stated previously, our animal model of fetal undernutrition displays a phenotype that closely resembles that described in the human clinical setting as the metabolic syndrome or “Syndrome X” (33). Syndrome X is a multifaceted syndrome characterized by the clustering of insulin resistance and hyperinsulinemia, and is often associated with hypertension, obesity, glucose intolerance and type 2 diabetes (42). Despite the suggestion that insulin itself mediates the clinical linkage, the specific mechanisms underlying this syndrome remain poorly understood. Our data show that IGF-I therapy alleviates insulin resistance, hyperleptinemia and hypertension and may restore functional feedback between insulin and leptin following perturbations in the adipoinsular axis as a result of fetal programming. IGF-I therapy may also ameliorate obesity, hyperphagia and hypertension by differential regulation of downstream signalling networks via the IRS, RAS and IGF-I receptor signalling pathways by independent and complementary mechanisms.

Of particular clinical benefit is the potential use of IGF-I, analogues or ligands, in individuals who have been exposed to fetal programming but are otherwise essentially healthy (ie. prior to the development of the consequences of fetal programming, such as hypertension, obesity, hyperphagia, diabetes and other metabolic disorders). Indeed, it is conceivable that such therapy may reverse the effects of fetal programming.

The application of this invention to humans and other animals suffering from fetal programming due to the effects of undernutrition and IUGR will allow for prophylactic, and also direct, treatment regimes. As is readily apparent, review of maternal health (as is discussed herein) will allow clear indication of individuals who are likely to suffer from conditions associated with fetal programming later in life. Treatment, following identification of at risk individuals, is a simple and relatively cheap matter using accepted techniques. In animals other than humans similar opportunities arise. Application to production animals (sheep, cows, deer etc.) suffering from the effects of drought or similar environmental conditions, could mitigate the ongoing effects (e.g. on body weight, health etc) of the next generation of animals.

### Example 2

### Modulation of Angiotensin II Receptors

Animals were subjected to fetal programming as described above in Example 1. Subsequently, animals received either IGF-1 or its vehicle as described below.

At day 175, rats were weight matched (n=6 per group) and received either rh- IGF-I (Genentech Code #G117AZ, Batch c9831AY) or saline by osmotic minipump (Model 2002, Alzet Corp, Palo Alto, Calif. US). The dose was 3 μg/g/day for 14 days with a pump delivery rate of 5 μl per hour. The mean pump rate for the batch (Lot # 167258) of pumps used was 5.23±0.2 μl/hr. Pumps containing the IGF-I or saline solution were incubated in sterile saline for 4 hours at 37° C. prior to implantation. The osmotic pumps were implanted subcutaneously, under halothane anaesthesia, using a small incision made in the skin between the scapulae. Using a haemostat, a small pocket was formed by spreading apart the subcutaneous connective tissues. The pump was inserted into the pocket with the flow moderator pointing away from the incision. The skin incision was then closed with sutures. All animals (n=48) were housed individually for the duration of the study (WO 02/47714).

### Tissue Sections

Kidney tissues were collected from offspring of undernourished Wistar rat mothers. Tissues were collected from 8 experimental groups (n=6 per group), processes and then embedded in paraffin. Serial sections (5 μm, 4 sections per animals, 2 sections per slide) were cut using a microtome (Leica, model RM2035), placed on poly-L-lysine coated slides and left to dry overnight in an incubator (Wilton utility incubator, UTIL72860, 57° C.). After drying sections were deparaffinized with xylene and rehydrated with decreasing concentrations of alcohol through to PBS (0.01M) followed by distilled water. Tissues take from the following groups were examined:

UN control diet (UNC) saline

UN control diet (UNC) IGF-I treated

UN high fat diet (UNHF) saline

UN high fat diet (UNC) IGF-I treated

Immunohistochemistry for the ATIR was performed using the avidin-biotin (ABC) method for immunostaining of paraffin embedded sections (Vectastain Elite ABC kit, Vector Laboratories, USA). In brief, 5 μm sections were deparaffinized and treated with 1% H₂O₂ in methanol for 30 minutes at room temperature to inhibit endogenous peroxidase activity. Following this sections were washed in 0.01M PBS (pH 7.4) and incubated with 2.5% normal goat serum in 0.01M PBS (pH 7.4) containing 0.1% BSA (Lot 49284123, Roche). Sections were then incubated overnight at 4° C. in a humidified chamber with a polyclonal anti-AT₁R antibody (SC-597, Santa Cruz Biotechnology, SC, USA) diluted in 0.1M PBS with 0.1% BSA (Lot 49284123, Roche). A series of antibody titres were investigated and staining was optimised at a final primary antibody dilution ranging in the order of 1:50 to 1:100. After further washing, sections were incubated for 2 hours at room temperature with a biotinylated secondary antibody (Goat anti-rabbit IgG-Biotin). After a further washing step sections were incubated for 1 hour at room temperature with a avidin-biotin peroxidase complex (ABC). Immunoreactivity was then detected by the addition of diaminobenzidine (DAB) (Sigma, Lot 94H3677) and H₂O₂ in milli-Q water. Sections were then washed in 0.01M PBS (pH 7.4) and counterstained with Gills haematoxylin, dehydrated and mounted.

Negative controls were performed by substituting the primary antibody with normal rabbit serum (Sigma, Lot 10H93113, G-0261) at a 1:200 dilution at 4° C. overnight. This was done to identify any non-specific binding of the secondary antibody.

### Evaluation of Sections

All sections were examined for differences in staining intensity and localisation of AT₁R staining with a light microscope at 400× magnification. Sections were analysed by an experienced observer blinded to the treatment groups to assess diet and treatment effects on receptor immunoreactivity. Sections were graded on a scale of 1 to 3, which ranged from low intensity (1), moderate intensity (2) through to high intensity staining (3).

### Statistical Analysis

Statistical analysis was carried out using the StatView statistical package (Version 5, SAS Institute, Cary, N.C., USA). Differences in means between groups were determined by three-way (Glomerular Structure) and two-way (AT₁R immunoreactivity) ANOVA. Interaction effects between the various factors (diet, treatment and/or programming) were calculated and results were illustrated as histograms. Values were expressed as mean±SEM. p<0.05 was taken as statistically significant.

### Results

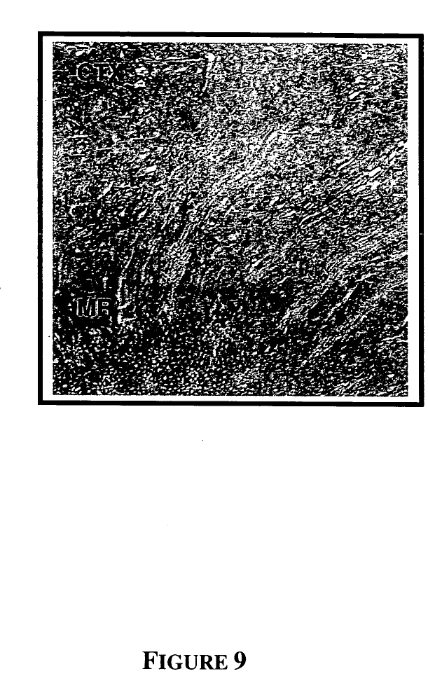

In normal animals, little AT₁R reactivity was found. in contrast, animals subjected to fetal programming exhibited greater immunoreactivity than unaffected animals. In particular, increased AT₁R immunoreacitivity was found in the glomeruli, in the proximal tubules and in the distal tubules. Immunohistochemistry for the AT₁R showed that postnatal hypercaloric nutrition did not affect the intensity and localisation of the AT₁R in the kidney of programmed animals. In contrast to this, all programmed animals that were treated with IGF-1 were observed to have a much lower intensity of staining of the AT₁R than their non-treated counterparts. This is depicted in the photographs below (refer to FIGS. 9-16). Regions of brown staining reflect AT₁R immunoreactivity.

FIG. 9 depicts a photomicrograph of an immunohistochemical section of a programmed kidney incubated with the AT₁R antibody. Localisation of the AT₁R immunoreactivity (brown staining) can be seen distinctly in the medullary region (MR). Slight immunoreactivity is also evident in the cortical region (CTX). (Mag 100×).

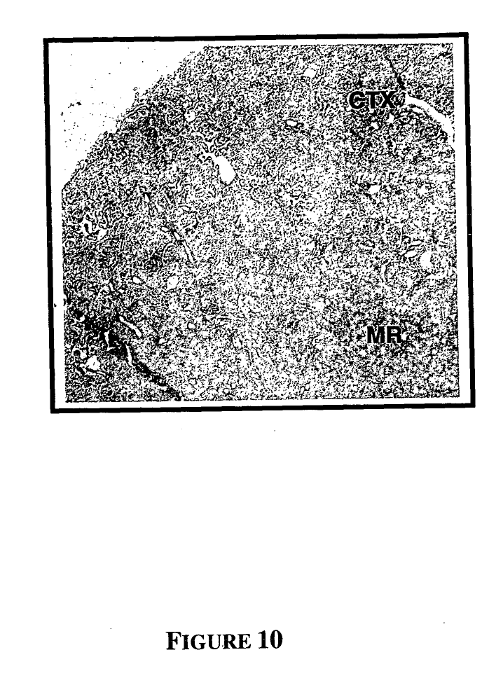

FIG. 10 depicts a photomicrograph of the negative control immunohistochemical kidney incubated with normal rabbit serum. No evidence of AT₁R immunoreactivity was observed. (Mag×50).

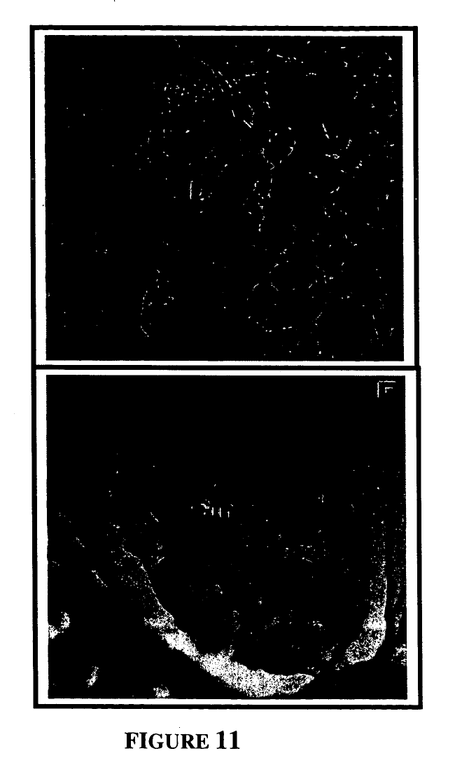

FIG. 11 depicts photomicrographs of an immunohistochemical section of a programmed kidney incubated with the AT₁R. Renal cortex demonstrates labelling throughout the glomeruli (Glm) and renal tubules, specifically the proximal (PT) and distal (DT) tubules. (A: mag 250×, B: mag 1000×).

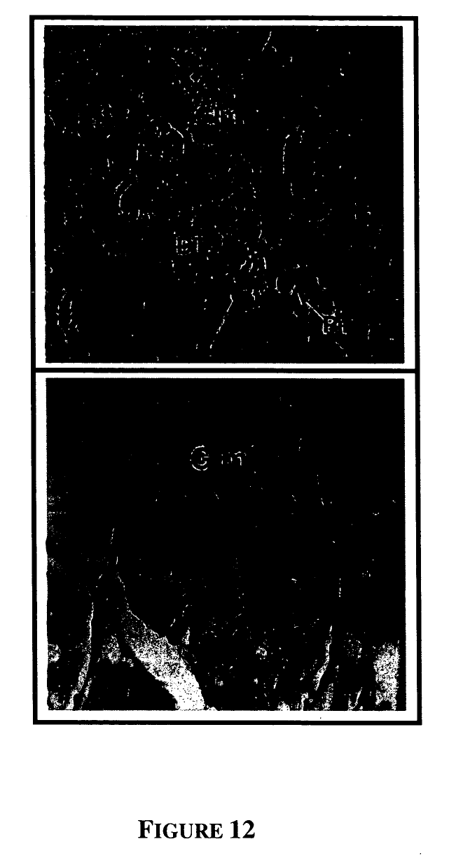

FIG. 12 depicts photomicrographs of an immunohistochemical section of a programmed kidney treated with IGF-1 incubated with the AT₁R. There is no evident labelling throughout glomeruli and renal tubules. (A: mag 250×, B: mag 1000×).

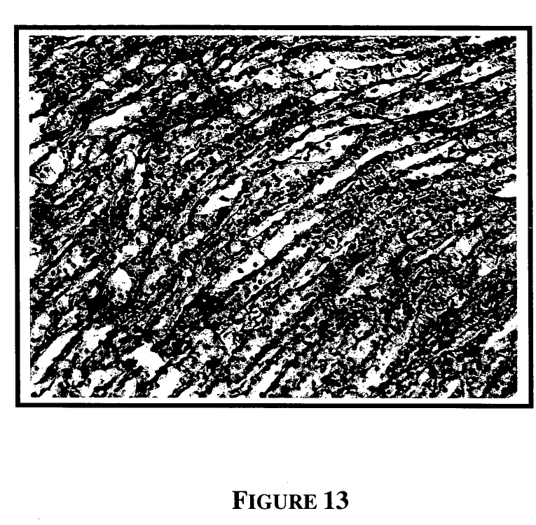

FIG. 13 depicts photomicrograph of an outer medullary immunohistochemical section of a programmed kidney incubated with the AT₁R. Distinct labelling can be seen in the renal tubules. (mag 250×)

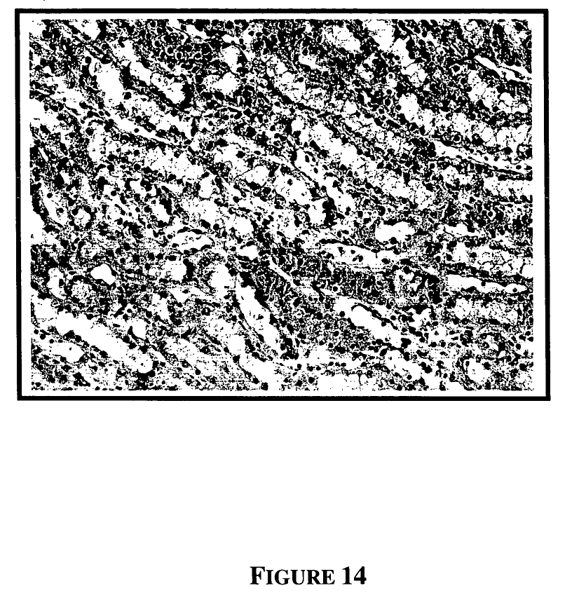

FIG. 14 depicts a photomicrograph of the outer medullary immunohistochemical section of a programmed kidney treated with IGF-1 and incubated with the AT₁R. Decreased AT1R immunoreactivity is seen (mag 250×).

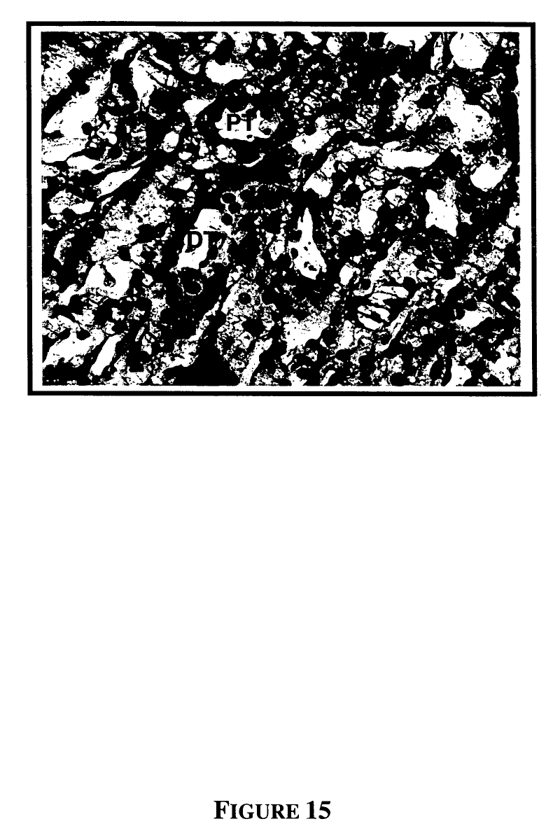

FIG. 15 depicts a photomicrograph of the outer medullary immunohistochemical section of a programmed kidney incubated with the AT₁R. Strong labelling of the proximal tubules is demonstrated with lesser staining within the distal tubules (mag 630×).

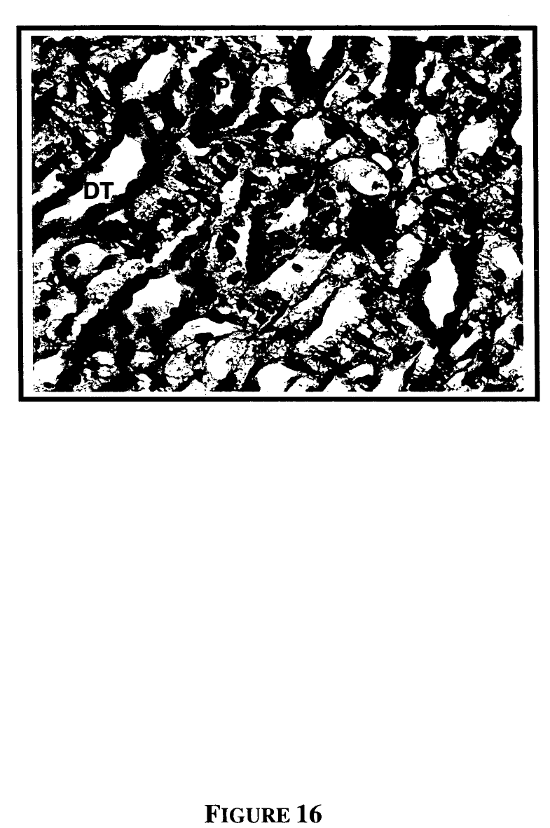

FIG. 16 depicts a photomicrograph of the outer medullary immunohistochemical section of a programmed kidney treated with IGF-1, incubated with the AT₁R. Little immunoreactivity is seen with both the proximal (T) and distal (DT) tubules (mag 630×).

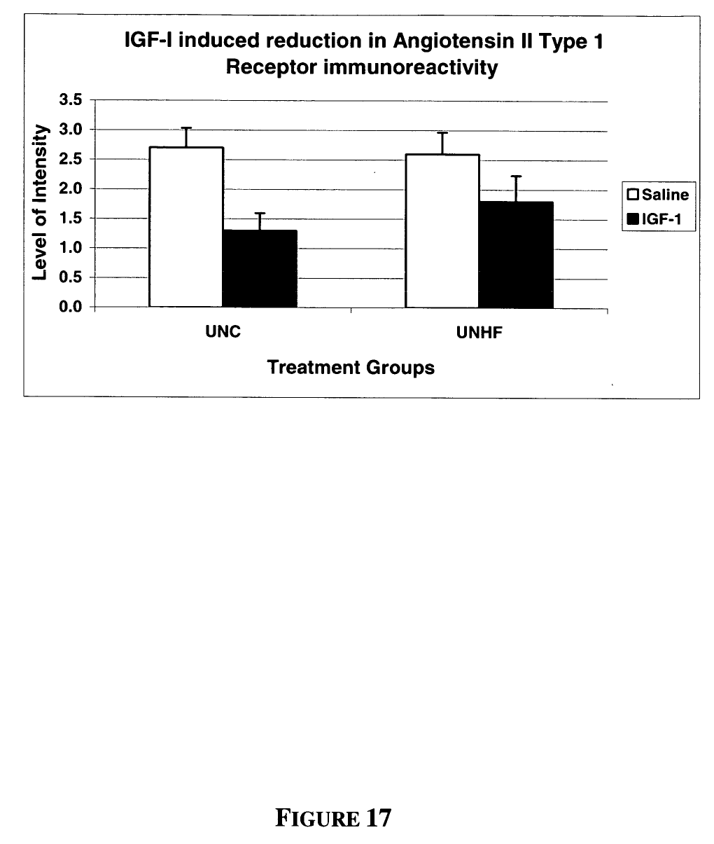

FIG. 17 depicts histograms showing the localisation and intensity of the AT₁R in the programmed offspring and Western blots showing levels of expression of AT₁R. Values are expressed as mean±SEM.

Table 4 below depicts results effects of diet and IGF-1 treatment on expression of AT₁R in kidneys of animals subject to fetal programming.

TABLE 4
Effects of Diet and IGF-1 Treatment on Expression of AT₁R
in Animals Subject to Fetal Programming

| Effect | p-value |
|---|---|
| Diet | 0.8672 |
| Treatment | 0.0284 |
| Interactions: | |
| Diet × Treatment | 0.3220 |

p < 0.05 is considered statistically significant.

There were no significant differences in the expression of the AT₁R as a consequence of postnatal hypercaloric nutrition. However IGF-1 treatment in programmed animals decreased the staining intensity and localisation of the AT₁R (p<0.05).

There is notable difference between the expression of the AT₁R protein in the kidney of programmed animals and their IGF-1 treated counterpart. There appears to be a link between the decreased expression of the AT₁R in IGF-1 treated programmed offspring and the finding that IGF-1 decreases blood pressure in programmed offspring.

### Discussion

IGF-1 treatment significantly (p<0.05) reduced the overall expression of the AT₁R protein in the kidneys of offspring subjected to fetal programming. This IGF-1-mediated reduction in AT₁R expression, suggests mechanisms by which angiotensin II formation can be reduced, consequently reducing blood pressure and providing an additional therapeutic mode for treatment of hypertension in animals subjected to fetal programming. In particular, we found that animals subjected to fetal programming exhibited greater expression of AT₁R immunoreacitivity than did normal animals. In particular, AT₁R immunoreactivity was increased in the glomeruli, in the proximal tubules and in the distal tubules.

Our results indicate that in addition to any effects on vascular AT₁R, IGF-1 decreases expression of AT₁R in locations in the kidney that contribute to hemostasis. In partucular, blood pressure is known to be increased by increases in either cardiac output and total arterial resistance. Therefore, treatments for hypertension include those that decrease total arterial resistance (e.g., nitroglycerin) by relaxing arteriolar smooth muscle. Because AT₁R can mediate increased vascular smooth muscle tone, increases in AT₁R expression can lead to vasoconstriction in situations in which ANG II is elevated. Similarly, by promoting the release of epinephrine and nor-epinephrine (agents that can activate alpha-adrenoreceptors on arteriolar smooth muscle), increased expression of AT₁R can increase blood pressure indirectly, through actions on the sympathetic nervous control of vascular tone. Moreover, because AT₁R activation can lead to increased vasopressin release, blood pressure can be increased by way of that mechanism also.

Conversely, decreasing AT₁R expression on vascular smooth muscle cells can lead to decreased potency of ANG II, and therefore, in situations in which ANG II levels are increased, such decreased expression can lead to decreases in the elevation in blood pressure normally observed in animals subjected to fetal programming. Thus, decreased expression of AT₁Rs can reduce the effects of sympathetic activation and vasopressin release, thereby indirectly resulting in reductions in abnormally high vascular tone and reductions in blood pressure.

In addition to effects on resistance vessels, our surprising findings support a substantial role of renal AT₁R receptors in regulation of blood pressure in animals subjected to fetal programming. Thus, fetal programming can lead to increased expression of AT₁R in the glomeruli, proximal tubules and the distal tubules. I the glomeruli, AT₁Rs are known to be associated with increased glomerular filtration, and in the proximal and distal tubules, expression of AT₁Rs are associated with increased sodium uptake by tubule cells from the lumen to the interstitial space of the kidney medulla and cortex. These effects can act in concert to promote sodium and water resorption by the kidney. As a result, blood volume is maintained in the face of hypertension. This apparent lack of a normal, negative feedback control over blood volume may account at least in part for the hypertension observed in animals subjected to fetal programming.

Therefore, treatment of animals subjected to fetal programming with IGF-1, IGF-1 analogs or derivatives thereof, by decreasing the expression of AT₁Rs at locations associated with alteration in water resorption by the kidney, IGF-1 therapy can result in better hemostasis in the face of hypertension. By decreasing expression of AT₁Rs at those sites in the kidney, IGF-1 can promote decreased blood volume, and thereby can decrease cardiac output. By decreasing cardiac output, IGF-1 can thereby decrease blood pressure. Although the above mechanisms are described as separate, it is well known that physiological control of blood pressure involves regulation of both total arterial resistance and cardiac output. Thus, the overall control of blood pressure is, in physiological circumstances, mediated via simultaneous action of at least the above two mechanisms.

It should be appreciated that the above mechanisms are only examples of possible mechanisms by which IGF-1 decreases blood pressure in animals subjected to fetal programming. Other possible mechanisms of action might also play roles in hemostasis in these animals, and all such mechanisms are considered to be part of this invention.

### REFERENCES

1. Woodall S M, Breier B H, Johnston B M, Gluckman P D 1996. A model of intrauterine growth retardation caused by chronic maternal undernutrition in the rat: effects on the somatotropic axis and postnatal growth. J Endocrinol 150:231-242

2. Woodall S M, Johnston B M, Breier B H, Gluckman P D 1996. Chronic maternal undernutrition in the rat leads to delayed postnatal growth and elevated blood pressure of offspring. Pediatr Res 40:438-443

3. Vickers M H, Breier B H, Cutfield W S, Hofinan P L, Gluckman P D 2000.

Fetal origins of hyperphagia, obesity and hypertension and its postnatal amplification by hypercaloric nutrition. Am J Physiol 279:E83-E87

4. Jackson A A, Langley-Evans S C, McCarthy H D 1996. Nutritional influences in early life upon obesity and body proportions. Ciba Foundation Symposium 201:118-129

5. Ravelli A C, van der Meulen J H, Osmond C, Barker D J, Bleker O P 1999. Obesity at the age of 50 y in men and women exposed to famine prenatally. Am J Clin Nutr 70:811-816

6. Anguita R M, Sigulem D M, Sawaya A L 1993. Intrauterine food restriction is associated with obesity in young rats. J Nutr 123:1421-1428

7. Jones A P, Pothos E N, Rada P, Olster D H, Hoebel B G 1995. Matemal hormonal manipulations in rats cause obesity and increase medial hypothalamic norepinephrine release in male offspring. Brain Res Developmental Brain Research. 88:127-131

8. Baker J, Liu J P, Robertson E J, Efstratiadis A 1993. Role of insulin-like growth factors in embryonic and postnatal growth. Cell 75:73-82

9. Powell-Braxton L, Hollingshead P, Warburton C, Dowd M, Pitts-Meek S, Dalton D, Gillett N, Stewart T A 1993. IGF-I is required for normal embryonic growth in mice. Genes & Development 7:2609-

10. Muaku S M, Thissen J P, Gerard G, Ketelslegers J M, Maiter 1997. Postnatal catch-up growth induced by growth hormone and insulin-like growth factor-I in rats with intrauterine growth retardation caused by maternal protein malnutrition. Pediatr Res 42:370-377

11. Froesch E R, Bianda T, Hussain M A 1996. Insulin-like growth factor-I in the therapy of non-insulin-dependent diabetes mellitus and insulin resistance. Diabetes & Metabolism 22:261-267

12. Dunger D B, Acerini C L 1997. Does recombinant human insulin-like growth factor-I have a role in the treatment of diabetes?. Diabetic Medicine 14:723-731

13. Boni-Schnetzler M, Hauri C, Zapf J 1999. Leptin is suppressed during infusion of recombinant human insulin-like growth factor I (rhIGF I) in normal rats. Diabetologia 42:160-166

14. Zenobi P D, Jaeggi Groisman S E, Riesen W F, Roder M E, Froesch E R 1992. Insulin-like growth factor-I improves glucose and lipid metabolism in type 2 diabetes mellitus. J Clin Invest 90:2234-2241

15. Tomas F M, Chandler C S, Coyle P, Bourgeois C S, Burgoyne J L, Rofe A M 1994. Effects of insulin and insulin-like growth factors on protein and energy metabolism in tumour-bearing rats. Biochem J 301:769-775

16. Donath M Y, Sutsch G, Yan X W, Piva B, Brunner H P, Glatz, Zapf J, Follath F, Froesch E R, Kiowski W 1998. Acute cardiovascular effects of insulin- like growth factor I in patients with chronic heart failure. J Clin Endocrinol Metab 83:3177-3183

17. Delafontaine P 1995. Insulin-like growth factor I and its binding proteins in the cardiovascular system. Cardiovasc Res 30:825-834

18. Blum W F, Ranke M B 1990. Use of insulin-like growth factor-binding protein 3 for the evaluation of growth disorders. Horm Res 34(suppl 1):31-37

19. Jones J I, Clemmons D R 1995. Insulin-like growth factors and their binding proteins: Biological actions. Endocr Rev 16:3-34

20. Unterman T G, Lascon R, Gotway M B, Oehler D T, Gounis A, Simmons R A, Ogata E S 1990. Circulating levels of insulin-like growth factor binding protein-i (IGFBP-1) and hepatic mRNA are increased in the small for gestational age (SGA) fetal rat. Endocrinology 127:2035-2037

21. Tapanainen P J, Bang P, Wilson K, Unterman T G, Vreman H J, Rosenfeld R G 1994. Maternal hypoxia as a model for intrauterine growth retardation: effects on insulin-like growth factors and their binding proteins. Pediatr Res 36:152-158

22. Blum W F, Breier B H 1994. Radioimmunoassays for IGFs and IGFBPs.

Growth Regulation 4:11-19

23. Breier B H, Vickers M H, Gravance C G, Casey P J 1996. Growth hormone (GH) therapy markedly increases the motility of spermatozoa and the concentration of insulin-like growth factor-I in seminal vesicle fluid in the male GH-deficient dwarf rat. Endocrinology 137:4061-4064

24. Hossenlop P, Seurin D, Segovia-Quinson B, Hardouin S, Binoux M 1986. Analysis of serum insulin-like growth factor binding proteins using Western-ligand blotting: use of the method for titration of the binding proteins and competitive binding studies. Ann Biochem 154:138-143

25. Gallaher B W, Breier B H, Oliver M H, Harding J E, Gluckman P D 1992. Ontogenic differences in the nutritional regulation of circulating IGF binding proteins in sheep plasma. Acta Endocrinol Copenh 126:49-54

26. Gargosky S E, Tapainen P, Rosenfeld R G 1994. Administration of growth hormone (GH), but not insulin-like growth factor-I (IGF-I), by continuous infusion can induce the formation of the 150-kilodalton IGF-binding protein-3 complex in GH-deficient rats. Endocrinology 134:2267-2276

27. Guler H-P, Zapf J, Scheiwiller E, Froesch E R 1988. Recombinant human insulin-like growth factor I stimulates growth and has distinct effects on organ size in hypophysectomised rats. Proc Natl Acad Sci USA 85:4889-4893

28. Tomas F M, Knowles S E, Owens P C, Chandler C S, Francis G L, Ballard F J 1993. Insulin-like growth factor-I and more potent variants restore growth of diabetic rats without inducing all characteristic insulin effects. Biochem J 291:781-786

29. Frick F, Oscarsson J, Vikman-Adolfsson K, Ottosson M, Yoshida N, Eden S 2000. Different effects of IGF-I on insulin-stimulated glucose uptake in adipose tissue and skeletal muscle. American Journal of Physiology—Endocrinology & Metabolism 278:E729-E737

30. Kieffer T J, Heller R S, Habener J F 1996. Leptin receptors expressed on pancreatic beta-cells. Biochem Biophys Res Commun 224:522-527

31. Seufert J, Kieffer T J, Leech C A, Holz G G, Moritz W, Ricordi C, Habener J F 1999. Leptin suppression of insulin secretion and gene expression in human pancreatic islets: implications for the development of adipogenic diabetes mellitus. J Clin Endocrinol Metab 84:670-676

32. Kieffer T J, Habener J F 2000. The adipoinsular axis: effects of leptin on pancreatic beta-cells. Am J Physiol 278:E1-E14

33. Reaven G M 1993. Role of insulin resistance in human disease (syndrome X): an expanded definition. Annual Review of Medicine 44:121-131

34. Shimomura I, Matsuda M, Hammer R E, Bashmakov Y, Brown M S, Goldstein J L 2000. Decreased IRS-2 and increased SREBP-1c lead to mixed insulin resistance and sensitivity in livers of lipodystrophic and ob/ob mice. Mol Cell Biochem 6:77-86

35. Szanto I, Kahn C R 2000. Selective interaction between leptin and insulin signalling pathways in a hepatic cell line. Proc Natl Acad Sci USA 97:2355-2360

36. Cohen B, Novick D, Rubinstein M 1996. Modulation of insulin activities by leptin. Science 274:1185-1188

37. Withers D J, White M F 2000. Perspective: The insulin signalling system—a common link in the pathogenesis of type 2 diabetes. Endocrinology 141:1917-1921

38. Zhao A Z, Zhao H, Teague J, Fujimoto W, Beavo J A 1997. Attenuation of insulin secretion by insulin-like growth factor 1 is mediated through activation of phosphodiesterase 3B. Proc Natl Acad Sci USA 94:3223-3228

39. Van Schravendijk C F, Heylen L, Van den Brande J L, Pipeleers D G 1990.

Direct effect of insulin and insulin-like growth factor-I on the secretory activity of rat pancreatic beta cells. Diabetologia 33:649-653

40. Agata J, Masuda A, Takada M, Higashiura K, Murakami H, Miyazaki Y,.

Shimamoto K 1997. High plasma immunoreactive leptin level in essential hypertension. American Journal of Hypertension 10: 1171 -1174

41. Aizawa-Abe M, Ogawa Y, Masuzaki H, Ebihara K, Satoh N, Iwai H, Matsuoka N, Hayashi T, Hosoda K, Inoue G, Yoshimasa Y, Nakao K 2000.

Pathophysiological role of leptin in obesity-related hypertension. J Clin Invest 105:1243-1252

42. Resnick L M 1993. Ionic basis of hypertension, insulin resistance, vascular disease, and related disorders. The mechanism of “syndrome X”. American Journal of Hypertension 6:123S-134S

43. Adamczak M, Zeier M, Dikow R, and Ritz E. (2002). Kidney and hypertension. Kidney International, 61 Supplement 80; S62-S67.

44. Allen M. et al. (2000). Localization and Function of Angiotensin AT₁ Receptors. American Journal ofHypertension. 13:31 S-38S. 45. Brody, Theodore M. et al. (1994). Antihypertensitive drugs. In Human Pharmacology: molecular to clinical. p. 159.

46. Centres for Disease Control and Prevention, National Center for Health Statistics, Division of Health Examination Statistics. Health, United States, 2001; 254.

47. Dalle S, Ricketts W, Imamura T, Vollenweider P, Olefsky JM. 2001. Insulin and Insulin-like Growth Factor -I Receptors Utilize Different G protein Signaling Coponents. J Biol. Chem.276; 15688-15695.

48. Delafontaine P. 1995. Insulin-like growth factor I and its binding proteins in the cardiovascular system. Cardiovasc Res 30:825-834.

49. Donath M Y, Sutsch G, Yan X W, Piva B, Brunner H P, Glatz, Zapf J, Follath F, Froesch E R, Kiowski W. 1998. Acute cardiovascular effects of insulin-like growth factor I in patients with chronic heart failure. J Clin Endocrinol Metab 83:3177-3183.

50. Froesch E. R, Zenbi P. D, Hussain M (1994) Metabolic and Therapeutic effects of Insulin-like growth factor I. Hormone research. 42, 66-71.

51. Henriksen E J, Jacob S, Kinnick T R, Teachey M K, Krekler M. 2001 Selective angiotensin II receptor receptor antagonism reduces insulin resistance in obese Zucker rats. Hypertension. 38(4) 884-890.

52. Hirschberg R, Adler S (1998) Insulin like growth factor system and the kidney:

physiological, Pathophysiological and Therapeutic Implications. American Journal ofKidney Disease. 31(6), 901-919.

53. Inagami T, Kambayashi Y, Ichiki T, Eguchi S, and Yamakawa T. (1999) Angiotensin receptors: molecular biology and signalling. Clinical and Experimental Pharmacology and Physiology 26; 544-549.

54. Kajstura J, Firdaliso F, Andreoli A M, Li B, Chimenti S, Medow S, Limana F, Nadal-Ginard B, Leri A, Aversa P. 2001 IGF-I overexpression inhibits the development of diabetic cardiomyopathy and angiotensin II-mediated oxidative stress. Diabetes 50(6):1414-24.

55. Kelmsdal T, et al. (1999). Effects of selective angiotensin II type 1 receptor blockade with losartan on arterial compliance in patients with mild essential hypertension. Blood Press; 8:214-219.

56. Langlois D, Ouali R, Berthelon M C, Derrien A, Saez J M. 1994. Regulation by growth factors of angiotensin II type-i receptor and the α subunit of GQ and G11 in bovine adrenal cells. Endocrinology 135(1);480-483.

57. Leri A, Liu Y, Claudio P P, et al. 1999a Insulin-like growth factor-I induces Mdm2 and down-regulates p53, attenuating the myocyte rennin-angiotensin system and stretch-mediated apoptosis. Am J Pathol 154:567-580.

58. Leri A, Liu Y, Wang X, Kajstura J, Malhotra A, Anversa P. 1999b Overexpression of insulin-like growth-i attenuates the myocyte renin-angiotensin system in transgenic mice. Circ Res 84:752-762.

59. Miyata et al ((1999) Distribution of angiotensin AT₁ and AT₂ subtypes in the rat kidney. American Journal ofphysiology: Renal Physiology. 46, F437-F446.

60. Navar et al ((1999) Intrarenal angiotensin II generation and renal effects of AT₁ receptor blockade. Journal of American Society of Nephrology, Apr Suppl 10, S266-72.

61. Nishimoto I. 1993. The IGF-II receptor system: a G protein-liked mechanism. Mol Reprod Dev35(4);398-406; discussion 406-407.

62. Sandberg K. J. H (2000). Kidney angiotensin receptors and their role in renal pathophysiology. Seminars in Nephrology. 20(5), 402-16.

63. Siragy H and Carey R. (2001). Angiotensin type 2 receptors: potential importance in the regulation of blood pressure. Current Opinion in Nephrology and Hypertension. 10:99-103.

64. Vickers M. H, Ikenasio B. A, Breier B. H (2001). IGF-1 treatment reduces hyperphagia, obesity, and hypertension in metabolic disorders induced by Fetal programming. Endocrinology. 142(9), 3964-3973.

65. Wailer D, Renwick A G, Hiller K. (2001). Hypertension. In Medical Pharmacology and Therapeutics. Saunders.

Where in the foregoing description reference has been made to specific components or embodiments of the invention having known equivalents then such equivalents are herein incorporated as if individually set forth.

Although this invention has been described by way of example and with reference to specific embodiments thereof it is to be understood that modifications or improvements may be made thereto without departing from the scope or spirit of the invention as described herein and in the appended claims.

We Claim:

1. A method for modulating the density and/or distribution of angiotensin II receptors in a mammal, comprising the step of administering an effective amount of an insulin-like growth factor-1 (IGF-1) compound sufficient to reduce antigiotensin II receptors in the kidney of said mammal.

2. The method of claim 1, wherein said IGF-1 compound is selected from the group consisting of IGF-1, IGF-2, des(1-3) IGF-1.

3. The method of claim 1 wherein the angiotensin II receptors are angiotensin II type 1 receptors and wherein their density, distribution, and potential for signal transduction are down-regulated.

4. The method of claim 1 wherein the angiotensin II receptors are angiotensin II type 2 receptors and wherein their density, distribution and potential for signal transduction are up-regulated.

5. The method of claim 1, wherein the mammal is human.

6. The method of claim 1, wherein said angiotensin II receptors are decreased in at least one tissue selected from kidney glomeruli, proximal tubules and distal tubules.

7. The method of claim 1, wherein the effective amount of said IGF-1 compound is administered in a form of a pharmaceutical composition including a pharmaceutically acceptable carrier thereof.

8. The method of claim 1, wherein the effective amount of IGF-1 compound is administered by way of administration of a replicable vehicle encoding for said IGF-1.

9. The method of claim 1, wherein the effective amount of IGF-1 compound is administered by intramuscular injection, subcutaneous injectdion, intraperintoneal injection or by implant.

10. The method of claim 1, wherein the said effective amount of IGF-1 compound is administered through an intravenous, transdermal, transmucosal, oral or epidural route.

11. The method of claim 1, wherein the effective amount of said IGF-1 compound is between 0.1 μg/kg/day and about 1 mg/kg/day.

12. A method for decreasing the expression of angiotensin II receptors in a mammal, comprising administering to said mammal an amount of a compound effective to increase the concentration of IGF-1 in said mammal.

13. The method of claim 12 wherein the increase of the concentration of IGF-1 or IGF-I analog is by about 0.1 μg/kg/day to about 1 mg/kg/day.

14. A method for reducing hypertension associated with increased expression of angiotensin II receptors in a mammal, comprising the step of administering an effective amount of an IGF-1 compound along with an effective amount of an inhibitor of angiotensin converting enzyme (ACE).

15. The method of claim 14, wherein said ACE inhibitor is selected from the group consisting of captopril, cilazapril, enalapril, fosinopril, imidapril, lisinopril, moexipril, perindopril, quinapril, ramipril and trandolapril.

16. A method for reducing hypertension associated with increased expression of angiotensin II receptors in a mammal, comprising the step of administering an effective amount of an IGF-1 compound along with an effective amount of an angiotensin II receptor antagonist.

17. The method of claim 16, wherein said angiotensin II receptor is selected from the group consisting of angiotensin II antagonist can be selected from a group that includes candesartan, irbesartan, losartan, telmisartan and valsartan.

18. A method for enhancing the antihypertensive and renoprotective properties of ACE inhibitors and angiotensin II antagonists comprising the step of administering to a mammal an effective amount of an insulin-like growth factor-I (IGF-I) compound, where an IGF-I compound comprises IGF-I, a biologically active IGF-I analog, a biologically active IGF-I mimetic, a compound that increases the concentration of IGF-I, or a compound that increases the concentration of IGF-I analogs in combination with the said ACE inhibitor or the said angiotensin II antagonist.
